(19) United States
(12) Patent Application Publication
(10) Pub. No.: US 2002/0104122 A1
(43) Pub. Date: Aug. 1, 2002

(54) Methods for producing a plant with enhanced resistance to pathogenic fungi

(75) Inventors: Makoto Kakitani, Takasaki-shi (JP); Naoyuki Umemoto, Yokohama-shi (JP); Isao Ishida, Tokyo (JP); Akihiro Iwamatsu, Yokohama-shi (JP); Masaaki Yoshikawa, Sapporo-shi (JP); Naoto Yamaoka, Sapporo-shi (JP); Youji Takeuchi, Sapporo-shi (JP); Kuniko Yoshikawa, Sapporo-shi (JP); Masashi Yoshikawa, Sapporo-shi (JP)

Correspondence Address: Stephen A. Bent, FOLEY & LARDNER, Washington Harbour, 3000 K Street, N.W., Suite 500, Washington, DC, 20007-5109, US

(73) Assignee: KIRIN BEER KABUSHIKI KAISHA

(21) Appl. No.: 09/816,127

(22) Filed: Mar. 26, 2001

Related U.S. Application Data
(63) Continuation-in-part of application No. 09/094,557, filed on Jun. 15, 1998, now Pat. No. 6,225,531.
Application No. 09/094,557 is a continuation-in-part of application No. 08/591,566, filed on Jul. 14, 1997, now abandoned.

(30) Foreign Application Priority Data
Jun. 17, 1994 (JP) 136100/1994
Dec. 15, 1995 (JP) 347823/1995

(51) Int. Cl. A01H 5/00

(52) U.S. Cl. 800/279

(57) ABSTRACT

This invention relates to a method for producing a plant with enhanced resistance to a pathogenic fungus, comprising transforming a plant with an expression vector comprising a DNA encoding a glucan elicitor receptor operably linked to a heterologous promoter, selecting a plant which has said DNA incorporated into a chromosome of said plant and which expresses said DNA, and recovering the selected plant. This invention also relates to a method for producing a progeny plant with resistance to a pathogenic fungus from said plant.

### CROSS-REFERENCES TO RELATED APPLICATIONS

This application is a continuation-in-part application of U.S. Patent Application Serial No. 09/094,557 filed June 15, 1998, which is a continuation-in-part application of U.S. Patent Application Serial No. 08/591,566 filed Feb. 14,1996.

### FIELD OF THE INVENTION

The present invention relates to a method for producing a plant or progeny thereof with enhanced resistance to pathogenic fungi.

### BACKGROUND OF THE INVENTION

It is known that plants synthesize and accumulate an antibiotic agent called phytoalexin in response to infection with pathogens (M. Yoshikawa (1978) Nature 257; 546). Some plant pathogens were found to have the substances that induce them to perform such a resistance reaction (N. T. Keen (1975) Science 187: 74), which are called “elicitors”. The biochemical process from the infection of plants with pathogens to the synthesis and accumulation of phytoalexin is believed to be as follows.

When the mycelium of a pathogen invades a plant cell, glucanase in the plant cell works so as to cleave polysaccharides on the surface of the pathogen mycelial wall, thereby liberating an elicitor. If the elicitor binds to a receptor in the plant cell, a second messenger which plays a role in signal transduction is produced. The signal transduction substance is incorporated in the nucleus of the plant cell and activates the transcription of the genes coding for phytoalexin synthesize enzymes to induce a phytoalexin synthesis. At the same time, the phytoalexin degradation is inhibited. As a result, phytoalexin is efficiently accumulated in the plant cell.

A phytoalexin playing an important role in the resistance of soybean is called glyceollin and its structure has been determined (M. Yoshikawa et al. (1978) Physiol. Plant. Pathol. 12: 73). Elicitor of soybean has a characteristic structure; it has β-1,6 linked glucan of various lengths as a principal chain from which β-1,3 linked glucan side chains are branched [J. K. Sharp et al. (1984) J. Biol. Chem. 259: 11321; M. Yoshikawa (1 990) Plant Cell Technology 1.2: 695]. A receptor specific for a glucan elicitor derived from a soybean pathogenic mold fungus Phytophthora megasperma f. sp. glycinea is believed to be a protein which plays an important role in the synthesis and accumulation of the antibiotic agent glyceollin. A method for the purification of the glucan elicitor receptor specific to this elicitor has been disclosed (E. G. Cosio et al., (1990) FEBS 264: 235, E. G. Cosio et at. (1992) Eur. J. Biochem. 204: 1115, T. Frey et al. (1993) Phytochemistry 32:543). However, the amino acid sequence of a glucan elicitor receptor has not been determined and the gene encoding the receptor is not yet known. If a gene coding for glucan elicitor receptor is found, it will be possible to create plants having resistance to pathogenic fungi by incorporating the gene into a chromosome of plants and expressing the glucan elicitor receptor in the plants. Thus, improvement of the productivity of agricultural products can be expected.

An object of the present invention is to provide a method for producing a plant transformed with a DNA molecule coding for a glucan elicitor receptor.

### SUMMARY OF THE INVENTION

As a result of intensive and extensive researches toward the resolution of the above assignment, the present inventors have succeeded in purifying a soybean root-derived glucan elicitor receptor, cloning a glucan elicitor receptor gene from a soybean cDNA library, transferring this gene into a tobacco plant and expressing it in the plant. Thus, the present invention has been achieved.

The present invention accordingly provides a glucan elicitor receptor having an amino acid sequence as substantially shown in SEQ ID NO:1.

The present invention also provides DNA molecules containing nucleotide sequences coding for a glucan elicitor receptor having an amino acid sequence as substantially shown in SEQ ID NO:1, and fragments thereof.

The present invention further provides DNA molecules containing nucleotide sequences coding for a glucan elicitor receptor which are incorporated in plasmid pER23-1, and fragments thereof.

The present invention still further provides vectors containing DNA molecules coding for a glucan elicitor receptor and plant cells transformed with DNA molecules coding for a glucan elicitor receptor.

Moreover, the present invention provides a method for producing a plant having resistance to pathogenic fungi, comprising transforming a plant with an expression vector comprising a DNA encoding a glucan elicitor receptor operably linked to a heterologous promoter, selecting a plant which has said DNA incorporated into a chromosome of said plant and which expresses said DNA, and recovering the selected plant.

In one embodiment of the invention, said DNA is derived from a plant.

In further embodiment of the invention, said DNA is derived from a dicotyledonous plant or a monocotyledonous plant.

In further embodiment of the invention, said DNA codes for a glucan elicitor receptor comprising (i) an amino acid sequence as shown in SEQ ID NO:1 or (ii) an amino acid sequence comprising residues 239-442 of SEQ ID NO:1.

In further embodiment of the invention, said method further comprises incorporating a DNA encoding a glucanase into a chromosome of said plant, and expressing said DNA in said plant.

In further embodiment of the invention, said DNA is derived from a plant.

In further embodiment of the invention, said DNA is derived from a dicotyledonous plant.

In further embodiment of the invention, the glucanase is of an extracellularly secreted type.

In further embodiment of the invention, said DNA encoding glucanase comprises a nucleotide sequence encoding an amino acid sequence as shown in SEQ ID NO:3 or SEQ ID NO:34.

The present invention further provides a plant having resistance to pathogenic fungi, characterized in that a DNA sequence coding for a glucan elicitor receptor has been transferred into the plant and the gene is expressed in it.

The present invention further provides a method for producing a progeny plant with resistance to a pathogenic fungus, comprising transforming an ancestral plant with an expression vector comprising a DNA encoding a glucan elicitor receptor operably linked to a heterologous promoter selecting a plant which has said DNA incorporated into a chromosome of said plant and which expresses said DNA, and obtaining progeny from the selected plant through cultivation and/or breeding.

The glucan elicitor receptor of the present invention is useful in the elucidation of resistance to fungi and the development of elicitor derivatives capable of inducing fungal resistance, and it can be used as an antigen for the production of antibodies against glucan elicitor receptors.

The DNA molecules of the present invention which contain nucleotide sequences coding for a glucan elicitor receptor and fragments thereof are useful as materials for establishing techniques for developing fungi-resistant plants. In other words, the DNA molecules of the present invention and fragments thereof may be introduced and expressed in various plants to enhance their fungal resistance.

Antibodies against the glucan elicitor receptor of the present invention, the DNA molecules of the present invention which contain nucleotide sequences coding for a glucan elicitor receptor, their mutants and antisense DNAs can be used in the studies of the elicitor-binding site of glucan elicitor receptor and signal transduction.

Furthermore, the information on the amino acid sequence of glucan elicitor receptor and the nucleotide sequence coding for glucan elicitor receptor can be used in studies on the elicitor-binding site of glucan elicitor receptor and on the signal transduction in which glucan elicitor receptor is involved.

The plants of the present invention are characterized by high resistance to fungi.

There exist elicitor receptor (ER) genes and ER proteins in a variety of plants including dicotyledon and monocotyledon, as demonstrated in Examples described later or as seen in the following literature:

1) FEBS Letters 431:405-410 (1998) discloses characterization and partial purification of an ER from parsley (Petroselinum cripum).

2) Biol. Chem. 381 :705-713 (2000) discloses that in the plant varieties French bean (Phaseolus vulgaris L. cv. Renia mora), Alfalfa (Medicago sativa L. var. Argon), Lupine (Lupinus multilupa L.), and soybean (Glycine max L. cv. 9007), homologous transcription products were detected by Northern hybridization using soybean GBP (β-glucan binding protein) as a probe; that in French bean (Phaseolus vulgaris L. cv. Renia mora), β-glucan binding protein which was isolated has 86% identity to the known soybean cDNA and has 85% identity plus 5% similarity to the protein encoded by the soybean cDNA; that β-glucan binding protein cDNA cloning was conducted in bean and soybean; and that β-glucan binding protein from soybean was expressed in a tomato cell.

3) BioEssays 20:569-576 (1998) discloses identification of elicitor-binding proteins and that Fabaceae plants (Pisum sativum, Medicago saliva, and Lupimus albus) have elicitor binding sites.

4) Physiol. Plant 98:365-374 (1996) discloses that H₂O₂ is generated via ER upon response of a plant cell to a pathogen, thereby conferring disease resistance to the plant.

It has further been suggested that there is a correlation between the ER gene or ER protein and the resistance on fungi.

In accordance with the present invention, integration of an ER gene into a wide range of plants results in a fungal resistance phenotype, upon expression of the ER gene. Moreover, elicitor receptors are known to exist in various plants, based on identification of high-affinity binding sites in plant membranes. For example, see Yoshikawa (1993) Plant Cell Physiol. 34: 1229. The publications cited above confirm the presence of elicitor receptors in these plants. By the same token, ER genes will be identified, pursuant to the inventors' approach described here, by the ability of such genes to hybridize to an oligonucleotide designed in light of the ER-encoding nucleotide sequence of SEQ. ID NO:2. This approach is illustrated in Examples 11 and 1 2, involving hybridization to a radiolabeled cDNA encoding the elicitor receptor from soybean.

Likewise, glucanase (“Glu”) is also known to exist in a variety of plants (Plant Physical. 93:673-682 (1990); Plant Mol. Biol. 20:609-618 (1992); and Crit. Rev. Plant SCI. 13:325-387 (1994)). With ER, Glu can also be used for producing fungus-resistant plants by transformation. Glu genes are described in, for example, Plant Physical. 93:673-682 (1990) and Plant Mol. Biol. 20:609-618 (1992), and they are known to exist in the other plants (e.g., tobacco and barley) in which Glu amino acid sequences are homologous to the amino acid sequence of soybean Glu (Plant Physical. 93:673-682 (1 990)) except for the plants of soybean and kidney bean which are both written in this specification. Furthermore, there is described the isolation of a partial cDNA clone or genomic clone for pea-derived β-1,3Glu in Plant Mol. Biol. 20:609-618 (1992), and this literature further describes that the deduced amino acid sequence of the deduced amino acid sequence of the pea β-1,3-Glu has 78% identity to that of bean β-1,3-Glu, 62 and 60% to two tobacco β-1,3-Glu, 57% to soybean β-1,3 -Glu, and 51% to barley β-1,3-Glu. This indicates that the identity of β-1,3-Glu is relatively high between these plants.

It is further known that the transcriptional level of a soybean Glu gene is important in disease resistance of soybean (Plant Physiol. 93:673-682 (1990) and β-1,3-Glu generally plays an important role in attack of fungi in monocotyledonous and dicotyledonous plants (Crit. Rev. Plant Sci. 13:325-387 (1994)).

As mentioned above, because ER and Glu, as well as genes encoding them, are known to exist in a variety of plants such as higher plants or dicotyledonous plants or monocotyledonous plants, a person skilled in the art can choose and use genomic DNA or cDNA encoding ER alone or ER and Glu for production of plants with resistance to fungi.

It is accordingly predicted that co-expression of the ER gene and the glucanase gene in plants will enable the plants to bear potent resistance to fungi. Construction of vectors (e.g., plasmid) comprising DNA encoding ER and Glu or fragments thereof can be performed according to procedures as taught in Molecular Cloning (supra). Furthermore, transforming a host plant to be provided with resistance to fungi, with the vectors can appropriately be performed, as well as expression and identification of ER and Glu in the host. The effect of the transformation on resistance to fungi can also readily be evaluated in the host plant by known fungus resistance tests.

### BRIEF DESCRIPTION OF THE DRAWINGS

FIG. 1 shows SDS-polyacrylamide gel electrophoresis patterns of three purification steps.

FIG. 2 shows the maps of plasmids pER23-1 and pER23-2.

FIG. 3 shows the procedure for constructing plasmid pKV1-ER23.

FIG. 4 shows a transient increase in intracellular Ca2²⁺concentration after the addition of an elicitor to cultured soybean cells.

FIG. 5 shows a transient increase in intracellular Ca2 ²⁺concentration after the addition of an elicitor to transformed cultured tobacco cells.

FIG. 6 shows elicitor-binding activities of full-or partial length glucan elicitor receptor expressed in E. coli.

FIG. 7 shows the inhibition of the binding of an elicitor with an elicitor binding protein in a soybean cotyledon membrane fraction by an antibody against an elicitor-binding domain.

FIG. 8 shows the inhibition of an elicitor-induced phytoalexin accumulation in soybean cotyledons by an antibody against an elicitor-binding domain.

FIG. 9 shows the resistance of transformed tobacco plants to P. nicotianae.

FIG. 10 presents photographs showing the resistance of transformed tobacco plants to R. solani.

FIG. 11 shows the resistance of transformed tobacco plants to R. solani.

FIG. 12 shows the resistance of transformed tobacco plants to P. nicotianae in an inoculation test using zoospores from P. nicotianae.

FIG. 13 shows the structure of plasmid pPG 1.

FIG. 14 shows the results of determination of the glucanase activity of a glutathione-S-transferase/kidney bean glucanase fusion protein.

FIG. 15 shows DNA blot analysis of elicitor receptor gene homologs in plants and bacterium. In FIG. 15A, total genomic DNAs (20μg each) extracted from 6 cultivar of soybean (i.e., Acme, Flambeau, Green Hormer as positive control, Harosoy, Harosoy 63, and Merit), were digested with EcoRI, resolved by electrophoresis on 1% agarose gel, and blotted onto Hybond N⁺membrane. The membrane was probed with ³²P -labeled cDNA of receptor gene. In FIG. 15B, total genomic DNAs prepared from 9 other species of plant and P. megasperma f. sp. glycinea race 9 were individually digested with EcoRI and resolved by electrophoresis on 1% agarose gel. DNA amounts were adjusted to correspond to haploid genome: soybean 20 μg; bean 11.4 μg; mung bean 10.4 μg; pea 40 μg; potato 28.6 μg; Arabidopsis 1 μg; tobacco 40 μg; rice 15.6 μg; maize 40 μg; and P. megasperma f. sp. glycinea 2 μg. Signal-like bands appeared in pea and potato lanes were artifacts caused by imperfect blotting or hybridization.

FIG. 16 shows an alignment comparison of nucleotide sequences between soybean GEBP and B25158 or B24124, and locations of primers. FIG. 16A shows the comparison of B25158 with soybeans GEBP. Homology of the nucleotide sequence of B25158 with that of soybean GEBP was 68%. FIG. 16B shows the comparison of nucleotide sequences between GEBP and B24124. Solid lines show B24124 primer-annealed positions. Homology of the nucleotide sequence of B24124 with that of soybean GEBP was 73%.

FIG. 17 shows an alignment comparison of nucleotide sequences between B25158 and At1. Homology of the nucleotide sequence of B25158 with that of At1 was 84%.

FIG. 18 shows an alignment comparison of the deduced amino acid sequence of B25158 with that of At1. Homology of the deduced amino acid sequence of B25158 with that of At1 was 92%.

FIG. 19 shows an alignment comparison of the deduced amino acid sequence of At1 with that of soybean GEBP. Homology of the deduced amino acid sequence of At1 with that of soybean GEBP was 67%.

### DETAILED DESCRIPTION OF THE PREFERRED EMBODIMENTS

Glucan elicitor receptor is a protein involved in the production of phytoalexins and which functions as a receptor for glucan elicitors derived from glucan, a cell wall component of fungi. Its function is to signal microsomes and nuclei to increase the phytoalexin content in cells; this function is effected through binding to a glucan elicitor generated by the cleavage of a part of mycelial walls of a pathogen by β-1,3-glucanase in plant cells when the pathogen, e.g. a microorganism of the genus Phytophthora has invaded into plant tissues.

The glucan elicitor receptor of the present invention has an amino acid sequence as substantially shown in SEQ ID NO:1. . The “amino acid sequence as substantially shown in SEQ ID NO:1” includes amino acid sequences as shown in SEQ ID NO: 1 in which there may be a deletion(s), replacement(s) or addition(s) of an amino acid(s), provided that they maintain the function of a glucan elicitor receptor.

The glucan elicitor receptor of the present invention can be produced, for example, by a partially modified Cosio's method (E.J.B. (1992) 204:1115). Briefly, the roots, leaves and stems of soybean, preferably variety green homer are homogenized and a membrane fraction is collected from the resulting slurry, purified by ion-exchange chromatography and further purified by affinity chromatography using an elicitor as a ligand. The elicitor used in the affinity chromatography is preferably derived from Phytophthora megasperma f. sp. glycinea race 1 (ATCC34566) because it shows incompatibility for Green Homer (i.e., resistance to the pathogen).

The amino acid sequence of the glucan elicitor receptor thus prepared can be determined as follows.

The purified glucan elicitor receptor is transferred on a PVDF membrane (Millipore Co.) by electroblotting and digested with lysylendopeptidase (AP-1). The fragmented peptides are recovered from the PVDF membrane and fractionated by reversed-phase HPLC (μ-Bondasphere 5 μC8). The peak fractions are analyzed with a gas-phase protein sequencer (Applied Biosystems Co.).

The glucan elicitor receptor of the present invention is useful in the elucidation of resistance mechanism of plants to fungi and the development of elicitor derivatives capable of inducing resistance to fungi, and it can be used as an antigen for the production of antibodies against glucan elicitor receptors.

The present invention encompasses DNA molecules containing nucleotide sequences coding for a glucan elicitor receptor, and fragments thereof. The DNA molecules of the present invention have preferably at least one stop codon (e.g., TAG) adjacent to the 3′ end.

More specifically, the present invention encompasses DNA molecules containing nucleotide sequences coding for a glucan elicitor receptor having an amino acid sequence as substantially shown in SEQ ID NO: 1, and fragments thereof. The “DNA molecules containing nucleotide sequences coding for a glucan elicitor receptor” include all degenerate isomers. The term “degenerate isomers” means DNA molecules coding for the same polypeptide with different degenerate codons. If a DNA molecule having the nucleotide sequence of SEQ ID NO:2 is taken as an example, a DNA molecule in which a codon for any amino acid, e.g., AAC for Asn is changed to a degenerate codon AAT is called a degenerate isomer. Examples of such degenerate isomers include DNA molecules containing the nucleotide sequence shown in SEQ ID NO: 2.

In another aspect, the present invention provides DNA molecules containing nucleotide sequences coding for a glucan elicitor receptor, which are incorporated in plasmid pER23-1, and fragments thereof. E. coli DH5 α EKB633 transformed with plasmid pER23-1 was internationally deposited with the National Institute of Bioscience and Human-Technology, the Agency of Industrial Science and Technology, on Jun. 15, 1994 under Accession Number FERM BP-4699 (1-3, Higashi 1-chome, Tsukuba-shi, Ibaraki-ken 305, JAPAN).

The DNA molecules of the present invention which contain nucleotide sequences coding for a glucan elicitor receptor and fragments thereof may optionally bind to an ATG codon for initiation methionine together with a translation frame in the upstream portion toward the 5′ end and also bind to other DNA molecules having appropriate lengths as non-translation regions in the upstream portion toward the 5′ end and the downstream portion toward the 3′ end.

The DNA molecules of the present invention which contain nucleotide sequences coding for a glucan elicitor receptor and fragments thereof can be present typically in the form of parts of constituents of plasmid or phage DNA molecules or in the form of parts of constituents of plasmid, phage or genomic DNA molecules which are introduced into microorganisms (particularly, bacteria including E. coli and Agrobacterizium), phage particles or plants.

In order to express stably the DNA sequences coding for a glucan elicitor receptor, or fragments thereof in plants, a promoter, a DNA molecule (ATG) encoding the initiation codon and a terminator may be added to the DNA sequences, or fragments thereof of the present invention in appropriate combinations. Examples of the promoter include the promoter of genes encoding ribulose-1,5-biphosphate carboxylase small subunit (Fluhr et al., Proc. Nati. Acad. Sci. USA (1986) 83:2358), the promoter of a nopaline synthase gene (Langridge et al., Plant Cell Rep. (1985) 4:355), the promoter for the production of cauliflower mosaic virus 19S-RNA (Guilley et al., Cell (1982) 30:763), the promoter for the production of cauliflower mosaic virus 35S-RNA (Odell et al., Nature (1985) 313:810) and the like. Examples of the terminator include the terminator of a nopaline synthase gene (Depicker et al., J. Mol. Appi. Can. (1982) 1:561) and the terminator of an octopine synthase gene (Gielen et al., EMBO J. (1984) 3:835).

The DNA molecule containing a nucleotide sequence coding for a glucan elicitor receptor can be obtained by a method comprising the steps of chemically synthesizing at least a part of the DNA molecule according to a conventional procedure of synthesis of nucleic acids and obtaining a desired DNA molecule froth an appropriate cDNA library using the synthesized DNA molecule as a probe by a conventional method, for example, an immunological method or a hybridization method. Some plasmids, various kinds of restriction enzymes, T4 DNA ligase and other enzymes for use in the above method are commercially available. The DNA cloning, the construction of plasmids, the transfection of a host, the cultivation of the transfectant, the recovery of DNA molecules from the culture and other steps can be performed by the methods described in Molecular Cloning, J. Sambrook et al., CSH Laboratory (1989), Current Protocols in Molecular Biology, F. M. Ausubel et al., John Wiley & Sons (1987) and others.

More specifically, the DNA molecules of the present invention which contain nucleotide sequences coding for a glucan elicitor receptor can be obtained as follows:

Two kinds of partial amino acid sequences are selected from the amino acid sequences of a glucan elicitor receptor. Primers consisting of combinations of all nucleotides which can encode the C-terminus of the selected partial sequence and primers consisting of combinations of all nucleotides which can encode the N-terminus of the selected partial sequence are prepared. These synthesized primers are used as mixed primers to perform two PCR using DNA molecules of an appropriate soybean cDNA library as a template. Subsequently, two amplified fragments of given lengths whose amplification is expected (these fragments correspond to DNA molecules encoding the above two partial amino acid sequences) are picked up and the nucleotide sequences thereof are determined. On the basis of the determined nucleotide sequences, a primer having nucleotide sequences coding for the C-terminus of an amino acid partial sequence positioned at the C-terminal side of the glucan elicitor receptor and a primer having nucleotide sequences coding for the N-terminus of an amino acid partial sequence positioned at the N-terminal side of the glucan elicitor receptor are synthesized. These two synthesized primers are used to perform a PCR using the DNA molecules of the above soybean cDNA library as a template. The resulting amplified fragments are used as probes to hybridize the aforementioned soybean cDNA library, thereby yielding DNA molecules containing nucleotide sequences coding for the glucan elicitor receptor.

The obtained DNA molecules containing nucleotide sequences coding for the glucan elicitor receptor can be sequenced by any known methods, for example, the Maxam-Gilbert method (Methods Enzymol., 65:499, 1980), a dideoxynucleotide chain termination method using M13 phage (J. Messing, et al., Gene, 19:269, 1982) and the like.

Since the results of various studies on glucan elicitor suggest that a glucan elicitor receptor plays an important role in resistance to fungi in plants, it is expected that the DNA sequences coding for glucan elicitor receptor, or fragments thereof of the present invention can impart fungal resistance to plants if they are introduced and expressed in plant cells (particularly higher plant cells) which have no glucan elicitor receptor according to a conventional procedure. It has been proposed that fungi capable of infecting plants have generally suppressors, thereby acquiring an ability to suppress the fungal resistance of the plants. It is expected that new plants having resistance to fungi can be developed by introducing and expressing the DNA sequences coding for glucan elicitor receptor, or fragments thereof of the present invention such that the glucan elicitor receptor works or by modifying the DNA molecules or fragments thereof or by regulating their expression levels.

Moreover, if the DNA sequences coding for a glucan elicitor receptor, or fragments thereof of the present invention are introduced and expressed in plant cells, particularly in higher plant cells, together with fungal resistance enhancing genes or characters such as the gene of glucanase which imparts fungal resistance to plants, it is expected that higher fungal resistance can be imparted to plants than in the case where the gene of glucanase is introduced. Specific examples of a DNA molecule containing a nucleotide sequence coding for a glucanase include a DNA comprising a nucleotide sequence coding for a glucanase having an amino acid sequence as substantially shown in SEQ ID NO: 3 or 34. The “DNA comprising a nucleotide sequence coding for a glucanase” is intended to include all degenerate isomers. As a specific example of such degenerate isomers, a DNA comprising the nucleotide sequence as shown in SEQ ID NO: 4 or 33 may be mentioned.

Vectors used for introducing the DNA sequences coding for a glucan elicitor receptor, or fragments thereof may be constructed such that the glucan elicitor receptor can be stably expressed in plants. More specifically, a promoter, a DNA molecule encoding the initiation codon (ATO) and a terminator may be added to the DNA sequences coding for a glucan elicitor receptor, or fragments thereof in appropriate combinations. Examples of the promoter include the promoter of genes encoding ribulose-1,5-biphosphate carboxylase small subunit (Fluhr et al., Proc. Natl. Acad. Sci. USA (1986) 83:2358), the promoter of a nopaline synthase gene (Langridge et al., Plant Cell Rep. (1 985) 4:355), the promoter for the production of cauliflower mosaic virus 19S-RNA (Guilley et al., Cell (1982) 30:763), the promoter for the production of cauliflower mosaic virus 35S-RNA (Odell et al., Nature (1985) 313;810) and the like. Examples of the terminator include the terminator of a nopaline synthase gene (Depicker et al., J. Mol. Appl. Gen. (1982) 1:561) and the terminator of an octopine synthase gene (Gielen et al., EMBO J. (1984) 3:835).

The DNA molecules containing nucleotide sequences coding for a glucan elicitor receptor can be introduced into plant cells by any usual known methods, for example, the method described in “Plant genetic transformation and gene expression; a laboratory manual”, J. Draper, et al. eds., Blackwell Scientific Publications, 1988. Examples of the methods include biological methods such as those using viruses or Agrobacteria and physicochemical methods such as electroporation, a polyethylene glycol method, microinjection, particle gun method, dextran method and the like.

When the plant to be transformed is a dicotyledonous plant, the method using Agrobacterium is generally preferable. When the plant to be transformed is a monocotyledonous plant or a dicotyledonous plant that is not susceptible to infection with Agrobacterium, a physical/chemical method such as electroporation is preferable. As a plant material into which a DNA of interest is to be transferred, an appropriate material may be selected from leaves, stems, roots, tubers, protoplasts, calli, pollen, seed embryos, shoot primordia, etc. according to the method of transfer or the like.

When a DNA of interest is to be transferred into cultured plant cells, protoplasts are generally used as a material and the DNA is transferred thereinto by a physical/chemical method such as electroporation, the polyethylene glycol method or the like. On the other hand, when a DNA of interest is to be transferred into plant tissues, leaves, stems, roots, tubers, calluses, pollen, seed embryos, shoot primordia or the like are used as a material; preferably, leaves or stems are used. The DNA is transferred into such plant tissues by a biological method using a virus or Agrobacterium or a physical/chemical method such as the particle gun method, microinjection or the like; preferably, a biological method using Agrobacterium is used.

In order to regenerate a plant from those plant tissues or plant cells into which a DNA sequence coding for a glucan elicitor receptor has been transferred, these transformed plants or cells may be cultured in a medium such as hormone-free MS medium, if they are derived from tobacco. The resultant seedlings which are rooting may be transferred to soil to give grown-up plants.

As plants which can be imparted resistance or enhanced resistance to pathogenic fungi by transferring a DNA sequence coding for a glucan elicitor receptor, or a fragment thereof and expressing the gene by the methods described above, plants which are susceptible to infection with pathogenic fungi containing glucan in the cell walls may be mentioned. Specific examples of these plants include, but are not limited to, solanaceous plants and leguminous plants. More specifically, these plants include, but are not limited to, tobacco, soybean, potato, rice, chrysanthemum and carnation.

As pathogenic fungi, those containing glucan in the cell walls are embraced by the present invention. Specific examples of the pathogenic fungi include, but are not limited to, the genera Phytophthora, Rhizoctonia, Pyricularia, Puccinia, Fusarium, Uromyces and Botrytis. More specifically, the pathogenic fungi include, but are not limited to, Phytophthora nicotianae, Rhizoctonia solani, Pyricularia oryzac, Puccinia horiana, Fusarium oxysporum, Uromyces dianthi and Botrytis cinerea.

According to the present invention, the DNA sequence coding for a glucan elicitor receptor, or fragments thereof transferred into a plant can be inherited to subsequent generations through seeds. Thus, the transferred DNA molecule is also present in those seeds which are formed from that pollen or ovaries of the plant of the invention, and the inherited character can be transmitted to the progeny. Accordingly, the plant of the invention into which a DNA sequence coding for a glucan elicitor receptor, or fragments thereof, has been transferred can be propagated through seeds without losing its resistance to pathogenic fungi. The plant of the invention can also be propagated by a mass propagation method using plant tissue culture or by conventional techniques such as cutting, layering, grafting, division, etc. without losing its resistance to pathogenic fungi.

Whether a transformed plant has resistance to fungi or not can be examined by the following test methods.

Resistance to a Phytophthora fungus can be assayed by inoculating a fungal mycelium directly into plants and observing the expansion of lesions. Alternatively, the resistance may be assayed by inoculating zoospores from the fungus and observing the formation and expansion of lesions.

Resistance to a soil fungus can be assayed by mixing cultured fungal cells with soil, sowing seeds or setting plants on the soil, and observing a phenomenon of damping-off.

The present invention will now be explained in greater detail with reference to the following examples which are by no means intended to limit the scope of the present invention. In the following examples, a glucan elicitor receptor is abbreviated to “ER”.

### EXAMPLES

Example 1

Purification of soybean root-derived glucan elicitor receptor.

1) Measurement of glucan elicitor binding activity of ER.

A complex of an elicitor (average molecular weight: 10,000) and tyramine (Tokyo Kasei Kogyo Co., Ltd.) was synthesized by the method of Xong-Joo Cheong (The Plant Cell (1991) 3: 127). The elicitor-tyramine complex was labelled with iodine using chloramine T.

A sample (protein amount <500 μg) was suspended in 500 μ1 of an assay buffer (50 mM Tris-HCl pH7.4, 0.1 M saccharose, 5 mM MgCl_{2,}1mM PMSF and 5 mM EDTA) and incubated at 0° C. for 2 hours. The iodine-labelled elicitor-tyramine complex in an amount of 7.0 nM (70 Ci/mmol, the number of moles is calculated on the assumption that the molecular weight of the elicitor is 10,000, and this applies to the following description) was added to the suspension and the mixture was incubated at 4° C. for 2 hours. The reaction solution was filtered through Whatman GF/B as treated with a 0.3% aqueous solution of polyethylene imine for at least 1 hour. The residue was washed 3 times with 5 ml of an ice-cold buffer (10 mM Tris-HCl pH 7.0, 1 M NaCI, 10 mM MgCl₂). The radioactivity retained on the filter was counted with a gamma counter (count A). In order to eliminate the effect of non-specific binding, the same procedure as above was performed except that 17 μM of the elicitor was added to the same sample, the mixture was suspended in the assay buffer and the suspension was incubated at 0° C. for 2 hours. The obtained count was subtracted from the count A to give a count (Δ cpm) of elicitor-specific binding.

The resulting count (Δ cpm) was divided by the total number of counts and then multiplied by the total amount of elicitor used in the experiment to calculate the amount of the elicitor-binding protein (in moles).

The purity of ER was checked by the above method.

2) Purification of soybean root-derived ER.

Soybean (Glycine max cv. Green Homer) seeds (Takayama Seed Co.) were cultured on vermiculite for 1 week and then aquicultured for 15 days to harvest roots (about 40 kg, wet weight). The harvested roots were stored at −80° C. until they were used for the purification of ER. An ice-cold buffer (25 mM Tris-HCl pH 7.0, 30 mM MgCl_{2,} 2 mM dithiothreitol, 2.5 mM potassium metabisulfite and 1 mM PMSF) was added to the roots (2 kg, wet weight) in an amount of 1.25 L and the mixture was homogenized with a Waring Blender for 2 minutes.

The resulting slurry was filtered through a Miracloth (Calbiochem Co.) and the filtrate was centrifuged at 9,000 rpm at 4° C. for 15 minutes, The supernatant was ultracentrifuged at 37,000 rpm at 4° C. for 20 minutes. The precipitate was suspended in 160 ml of an ice-cold buffer (25 mM Tris-HCl pH 7.4, 0.1 M sucrose, 5 mM MgCl_{2,}1 mM PMSF and 5 mM EDTA) to give a membrane fraction. An ampholytic detergent ZW3-1 2 (Boehringer Co.) was added to the membrane fraction to give a final concentration of 0.25% for solubilization of ER from the membrane fraction and the mixture was stirred at 8° C. for 30 minutes. The resulting mixture was ultracentrifuged at 37,000 rpm at 4° C. for 20 minutes to collect the supernatant containing the solubilized ER (soluble fraction). The soluble fraction (165 ml) was dialyzed against 21 of a buffer (50mM Tris-HCl pH 8.0, 0.2% ZW3-12, 4° C.) 4 times. Five milliliters of Protrap (Takara Shuzo Co., Ltd.) was added to the sample and the mixture was stirred at 8° C. for 30 minutes to remove proteases from the sample and to stabilize ER. The resulting mixture was centrifuged at 2,800 rpm at 4° C. for 2 minutes to collect the supernatant. The obtained supernatant (160 ml) was concentrated to about 50 ml using an ultrafiltration membrane YM-10 (Amicon) and the concentrate was dialyzed against an A buffer (50 mM Tris-HCl pH 8.0, 0.1 M sucrose, 5 mM MgCl_{2,}1 mM PMSF, 5mM EDTA and 0.2% ZW3-12, 4° C.).

The dialysate was applied to Q-Sepharose HP 26/10 (Pharmacia) and ER was eluted in a linear gradient of 0-1 M NaCl (Q-Sepharose active fraction). The ER was eluted at a NaCl concentration of about 0.45 M. The Q-Sepharose active fraction was diluted 3 folds with A buffer and the diluted fraction was applied to Mono Q 10/10 (Pharmacia Co.). The ER was eluted in a linear gradient of 0-1 M NaCI (Mono Q active fraction, 8 ml). The ER was eluted at a NaCl concentration of about 0.25 H.

The ER was purified with an affinity gel using an elicitor as a ligand as follows:

Elicitor was prepared according to N. T.; Keen with some modifications (Plant Physiol. (1983) 71: 460, Plant Physiol. (1 983) 71: 466). Briefly, the mycelial wall of pathogenic Phytophthora megasperma f. sp. glycinea race 1 (ATCC34566) was treated with zymolyase 1OOT (Kirin Brewery Co., Ltd.) to liberate an elicitor. After the treatment, Zymolyase 1 OOT was eliminated by the adsorption on CM-cellulose packed in a column. The resulting passage-through fraction was purified with a gel permeation chromatography G-75 (Pharmacia Co.) to collect an elicitor fraction whose average molecular weight was 10,000 Da. The glyceollin-inducing elicitor activity of the collected fraction was determined by the method of M. Yoshikawa (Nature (1978) 257:546). The addition of 8 μg of the elicitor to soybean cotyledons resulted in the induction of about 550/μg of glyceollin after 24 hours incubation.

In order to eliminate non-specific adsorption on the gel carrier, Mono Q active fraction was collected and stirred with about 33 mg of maltose-coupled glass gel (bed volume: about 100 μ1) at 8° C. for 1 hour. The gel was precipitated by centrifugation (1,000 rpm, 4° C., 2 minutes) to collect the supernatant (maltose-coupled glass gel passage-through fraction). The maltose-coupled glass gel was prepared by the method of A. M. Jeffrey et al. (Biochem. Biophys. Res. Commun., (1975) 62: 608). Briefly, 120 mg of maltose and 6 g of Glass Aminopropyl (Sigma Co.) were suspended in 36 ml of H₂O and the suspension was stirred at room temperature overnight. To the resulting suspension was added 36 ml of ethanol. Immediately thereafter a solution of sodium borohydride (864 mg) in ethanol (72 ml) was added to the mixture. The resulting mixture was sonicated for 2 minutes and stirred at room temperature for 5 hours. Water (288 ml) was added to the reaction mixture and the resulting mixture was cooled with ice and adjusted to pH 5.6 with acetic acid. The gel was washed with about 1.8 L of H₂O to remove the free maltose. Maltose contained in the washing solution was determined quantitatively by the method of J. H. Roe (J. Biol. Chem. (1955) 212:335) using an anthrone reagent. An amount of the gel-coupled maltose was estimated from the amount of the maltose contained in the washing solution. As a result, it was found that 60 mg of maltose was coupled to 6 g of Glass Aminopropyl.

About 17 mg of the elicitor-coupled glass gel (bed volume: about 50 μ1) was added to 8 ml of the maltose-coupled glass gel passage-through fraction and the mixture was stirred gently at 8° C. overnight. The gel was collected by centrifugation (1,000 rpm, 4° C., 2 minutes) and washed with 2 bed volumes of A buffer 2 times. The gel was washed additionally with 4 bed volumes of 0.1% SDS 3 times to collect gel-coupled ER (elicitor-coupled glass gel eluted fraction). The elicitor-coupled glass gel was prepared by the method of A. M. Jeffrey et al. (Biochem. Biophys. Res. Commun. (1975) 62: 608). Briefly, elicitor (37 mg) and Glass Aminopropyl (490 rag) were suspended in 6 ml of H₂O and stirred at room temperature overnight. Ethanol (6 ml) was added to the suspension and a solution of sodium borohydride (144 mg) in ethanol (12 ml) was added immediately thereafter. The mixture was sonicated for 2 minutes and stirred at room temperature for 5 hours. To the resulting mixture was added 48 ml of H₂O. The mixture was cooled with ice and adjusted to pH 5.6 with acetic acid. The free elicitor was determined quantitatively with an anthrone reagent. The amount of the gel-coupled elicitor was estimated from the amount of the free elicitor contained in the washing solution. As a result, it was found that 34 mg of the elicitor was coupled to 490 mg of Glass Aminopropyl.

The protein and ER amounts in the above steps for purification are summarized in Table 1.

Table 1. Protein and ER Amounts in the Steps for Purification.

(Soybean roots weighing 40 Kg. on a wet basis used as a starting material)

| | Protein (mg) | ER (pmol) |
|---|---|---|
| Membrane Fraction | 17900 | 30 |
| Soluble Fraction | 2000 | 214 |
| Q-Sepharose Active Fraction | 190 | 205 |
| Mono Q Active Fraction | 49 | 233 |
| Maltose-Coupled Glass Gel | 45 | 220 |
| Passage-through Fraction | | |
| Elicitor-Coupled Glass Gel | 0.004* | 45 |
| Eluted Fraction | | |

*: Estimated from the band intensity obtained by silver stain after SDS-PAGE.

Estimated from the band intensity obtained by silver stain after SDSPAGE.

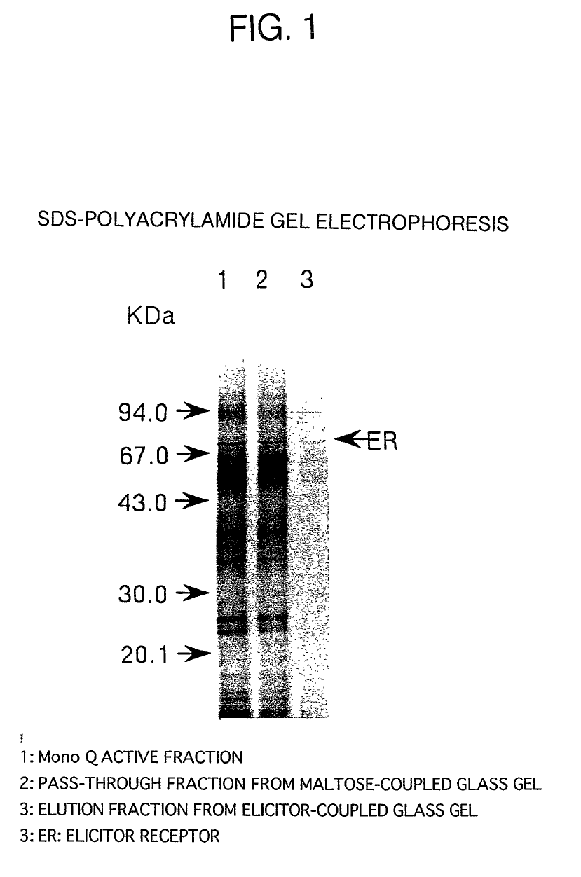

The Mono Q active fraction, passage-through fraction from maltose-coupled glass gel and eluted fraction from elicitor-coupled glass gel (μ1 each) were electrophoresed on an electrophoretic gradient gel, SDS-PAGE plate 10/20 (Daiich Kagaku Yakuhin Co.) and stained with silver (Daiich Kagaku Yakuhin Co.) The electrophoresis patterns are shown in FIG. 1. In FIG. 1, lane 1 is the Mono Q active fraction, lane 2; the passage-through fraction from the maltose-coupled glass gel and lane 3; the eluted fraction from the elicitor-coupled glass gel. FIG. 1 shows that the ER bands were detected at a molecular weight of about 70,000 Da.

The protein of about 70,000 in molecular weight was labelled with iodine-125 by using a ¹²⁵l-labelled complex of a photoaffinity reagent SASD (Pierce Co.) and the elicitor. The SDS-PAGE band of the membrane fraction was transferred on a PVDF membrane by western blotting and incubated with the same ¹²⁵l-labelled elicitor as used in measuring the elicitor-binding activity of ER on the PVDF membrane so that the protein of about 70,000 Da in molecular weight was labeled with iodine-125. These facts reveal that the protein of about 70,000 Da in molecular weight had an elicitor-binding activity.

About 4μg of ER was purified from about 40 kg by wet weight of the soybean root by the above method.

3) Analysis of ER-fragmented peptides

The ER was fragmented by protease digestion to peptides. The amino acid sequences of the fragmented peptides were determined by the method of lwamatsu (Akihiro lwamatsu, Seikagaku (1991) 63: 139, A. lwamatsu, Electrophoresis (1992) 13: 142). A solution of the ER purified by the above method was concentrated to about 100 a μ with Centricon-30 (Amicon Co.) and subjected to a 10-20% polyacrylamide SDS electrophoresis. The resulting protein bands were transferred on a PVDF membrane (Millipore Co.) with an electroblotting apparatus (Sartrius Co.). The bands transferred on the PVDF membrane were stained with 0.1% Ponceau S (Sigma Co.) 1% acetic acid. The main band of 70,000 Da in molecular weight was sectioned and decolored with 0.5 mM NaOH. This band was reductively Scarboxymethylated. Lysylendopeptidase (AP-1) was added to the resulting band at an enzyme:substrate (mol:mol) ratio of 1:100 and the mixture was reacted at 30° C. for 16 hours. The resulting fragmented peptides were applied to a μ-Bondasphere 5μC8-300 Å (2.1×150 mm, Waters) column equilibrated with 98% solvent A and 2% solvent B and eluted in a 2-50% linear gradient of solvent B for 30 minutes at a flow rate of 0.25 ml/minute (solvent A: 0.05% TFA solution, solvent B: 0.02% TFA in 2-propanol:acetonitrile=7:3 (v/v)). Eluted peptides were detected by absorbance at 214 nm and each peak fraction was collected manually. The obtained peak fractious were analyzed with a gas-phase protein sequencer (Model 470A of Applied Biosystems). As a result of analysis of all the peak fractions obtained, the following amino acid sequences of the fragmented peptides were clearly determined.

#1 Val Asn lie Gin Thr Asn Thr Ser Asn lie Ser Pro Gin (N-terminus) (SEQ XDNO:5)

#5:Lys Ser lie Asp Gly Asp Leu Val Gly Val Val Gly Asp Ser (SEQ ID NO:6)

#6: Lys Tyr Lys Pro Gin Ala Tyr Ser lie Val Gin Asp Phe Leu Asn Leu Asp (SEQ ID NO:7)

#7:Lys Thr Asp Pro Len Phe Val Thr Trp His Ser lle Lys (mix sequence) (SEQ ID NO:8)

### EXAMPLE 2

Cloning of soybean ER gene

l) Preparation of soybean RNA

Soybean (Glycine-max Cv. Green Homer) seeds (Takayama Seed Co.) were cultured on vermiculite for 1 week and aquicultured for 15 days to harvest roots (about 40 kg, wet weight). A portion of the harvested roots was stored at −80° C. until it was used. Total RNA was obtained by the method of Ishida (Cell Technology Laboratory Manipulation Manual, Kodansha Scientific). Briefly, the stored roots (28.5 g, wet weight) were ground on a mortar while adding liquid nitrogen. To the obtained powder, 35.6 ml of a GTC solution held at 65° C. was added and the mixture was homogenized with a Waring Blender. The resulting suspension was centrifuged at 6,000 rpm at room temperature for 15 minutes to collect 40 ml of the supernatant. The supernatant was layered gently on a cushion solution of cesium in a centrifuge tube and centrifuged at 35,000 rpm at 25° C. for 20 hours. The resulting precipitate was dissolved in 9 ml of TE/0.2% SOS. After phenol/chloroform extraction was conducted 2 times, total RNA (4.37 mg) was recovered by ethanol precipitation.

The obtained total RNA (2.2 mg) was used for purification with oligotex dT30 (Japan Roche Co.) according to the manual and then 68 μg of purified poly(A)+RNA was obtained.

2) Preparation of soybean cDNA library

cDNA molecules were synthesized from 5 μg of the poly(A) ^{+R} with a cDNA synthesis kit (Pharmacia Co.) according to the manual. The synthesized cDNA fragments were ligated to lambda phage vector λgt10 (Stratagene Co.) with T4 ligase (Takara Shuzo Co., Ltd.).

Gigapack (Stratagene Co.) was used to package a DNA mixture into the phage particles to prepare a soybean cDNA library of about 1.5×10⁶ pfu. The library was amplified to 1 60 ml of a soybean cDNA library of 1.6×10¹¹ pfu/ml.

Total DNA in the cDNA library was prepared as follows:

Chloroform/iso-amylalcohol (24:1) was added to 500 μ1 of a phage solution (1.6×10¹¹ pfu/ml) in an equal amount. The mixture was shaken for 30 seconds and centrifuged to collect the aqueous layer. The aqueous layer was extracted again with chloroform/isoamylalcohol (24:1). To the resulting aqueous layer were added 5 μ1 of a 3 M sodium acetate solution (pH 5.4) and 125 μ of ethanol and the mixture was centrifuged to collect the precipitate. The precipitate was washed with a 70% ethanol solution and dissolved in a 10 mM Tris-HCl Solution (pH 8) containing 1 μg/ml RNAse A (Sigma Co.). This solution was used as a PCR template.

3) Amplification and Cloning of Soybean ER cDNA Fragments by PCR

The following four oligodeoxynucleotides (mixed primers U5, U7, U10 and U12) were synthesized with an automatic nucleic acid synthesizer (Model 394 of Applied Biosystems Co.) on the basis of the amino acid sequences of the fragmented peptides obtained in Example 1 (#5 and #6):

Primer US 5′-AARAGYATHGAYGGNGA-3′ (SEQ ID NO:9)

Primer U5 5-WRTCNCCNACNAC-3′ (SEQ ID NO:10)

Primer U 10 5′-GTNAAYAARATNCARAC-S′ (SEQ ID NO: 11)

Primer U12 5′-ARRTTNAGRAARTCYTC-3′ (SEQ ID NO:12)

(R:A/O, Y:CIT, W:A/T, H:A/C/T, N:AfQ/T/C)

The total DNA in 0.5 μg of the cDNA library was dissolved in 79 μ1 of distilled water. Either a combination of primers U5 and U7 or a combination of primers U10 and U12 (100 pmol each) and 0.5 μ of TaqDNA polymerase (Takara Shuzo Co., Ltd.) were added to 8 μ1 of 2.5 mM dNTP in 10 4C4 1 of a 10 ×PCR buffer (attached to Taq DNA polymerase of Takara Shuzo Co., Ltd.) to give a final amount of 100 μ1. PCR reaction was performed with a Gene Amp PCR System 9600 (Perkin-Elmer Co.) by 50 cycles of 1) denaturation at 94° C. ×30 seconds, 2) renaturation at 47° C. ×30 seconds and 3) extension at 72° C. ×1 minute. After the reaction, 15 μ1 of the reaction solution was electrophoresed on 15% polyacrylamide gel. The gel was stained with a 0.5 μg/ml ethidium bromide solution for 10 minutes. The bands showing specifically amplified fragments of 40 bp and 47 bp whose amplification was expected were sectioned while observing under UV light. The gel sections were ground with a plastic bar and eluted with an elution buffer (0.5 M ammonium acetate, 10 mM magnesium acetate, 1 mM EDTA and 0.1% SDS) overnight to collect a DNA-containing solution.

The collected DNA fragments were cloned into plasmid pT7Blue(R) with a pT7Blue T-Vector Kit (Novagene Co.). The obtained plasmids p#5-1, 2, and p#6-1, 2, 3, 4, 5, 6, 7, 8 and 9 were sequenced with a fluorescence automatic DNA sequencer (Model 373A of Applied Biosystems Co.). The results showed that the resulting amplified DNA fragments other than the primers also encoded the amino acid sequences of fragmented peptides #5 and #6.

The following two oligodeoxynucleotides (mixed primers U18 and U19) were synthesized with an automatic nucleic acid synthesizer on the basis of the results of the DNA sequencing.

Primer U18 5′-AAGTAYAAGCCRCAAGCCTATTCA-3′ (SEQ ID NO:13)

Primer U19 5′-ATCGCCRACAACMCCAA-B′ (SEQ ID NO:14)

(Y and R are as defined above, M:A/C)

The total DNA in 0.5 μg of the cDNA library was dissolved in 79 μ1 of distilled water. A combination of primers U18 and U19 (100 pmol each) and 0.5 μ1 of Taq DNA polymerase were added to 8 μ12.5mM dNTP in 10 μ1 of a 10 ×PCR buffer to give a final amount of 100 μ. PCR reaction was performed by 40 cycles of 1) denaturation at 94° C. ×30 seconds, 2) annealing at 52° C. ×30 seconds and 3) extension at 72° C. ×1 minute. Fifteen microliters of the reaction solution was electrophoresed on a 1% agarose gel.

The gel was stained with a 0.5 μg/ml ethidium bromide solution for 15 minutes. The band showing a specifically amplified fragment of about 540 bp was sectioned while observing under UV light. The gel section was treated with Gene Clean II (Bio101 Co.) to collect a DNA-containing solution.

The collected DNA fragment was cloned into plasmid pT7Blue(R) with a pT7Blue T-Vector Kit. The obtained plasmid p#5-#6 was sequenced with a fluorescence sequencer. The results showed that the amplified DNA fragment consisted of 539 bp and encoded not only the amino acid sequences of fragmented peptides #5 and #6 at the both sides, but also peptide #7 in the amplified portion.

4) Screening and Cloning of Library by Hybridization

Plasmid #5-#6 into which the ER cDNA fragment was cloned was digested with restriction enzymes BamHl and PstlA DNA fragment of about 540 bp was recovered and used as a probe. The recovered DNA fragment was labelled with [α−³²P] using a Megaprime DNA labelling system (Amersham) according to the manual and the reaction solution was used in a hybridization experiment.

A phage of the cDNA library was infected with E. coli C600 hfl (Invitrogen) and inoculated in a 10 mg/nb MgCl₂-containing L medium on plates of about 15 cm in diameter to form a total of about 1 1×10⁶ of plaques. The plaques were blotted on a nylon membrane (Hybond-N; Amersham). The membrane was reacted with the ³²P-dCTP labeled ER eDNA fragment and positive phages detected by autoradiography were screened again in the same way to give about 30 phage clones having different signal intensities. Clone λ ER23 having the longest inserted DNA fragment was selected.

The λ phage DNA molecule was purified with a LambdaSorb (Promega) from a solution of the positive clone a ER23 isolated in the hybridization experiment. Ten microliters of a 10 ×EcoRl cleavage buffer (restriction enzyme EcoRl 10 U) was added to 5 μg of the DNA solution to give a total amount of 100 μ1 and the mixture was reacted at 37° C. overnight. The reaction solution was electrophoresed on a 1% agarose gel. A band of about 2.3 kb was sectioned and treated with a Gene Clean II (Bio101Co.) to collect a DNA-containing solution. Vector pBluescriptII KS- (0.02 μg) (Stratagene) was cleaved with restriction enzyme EcoRl.

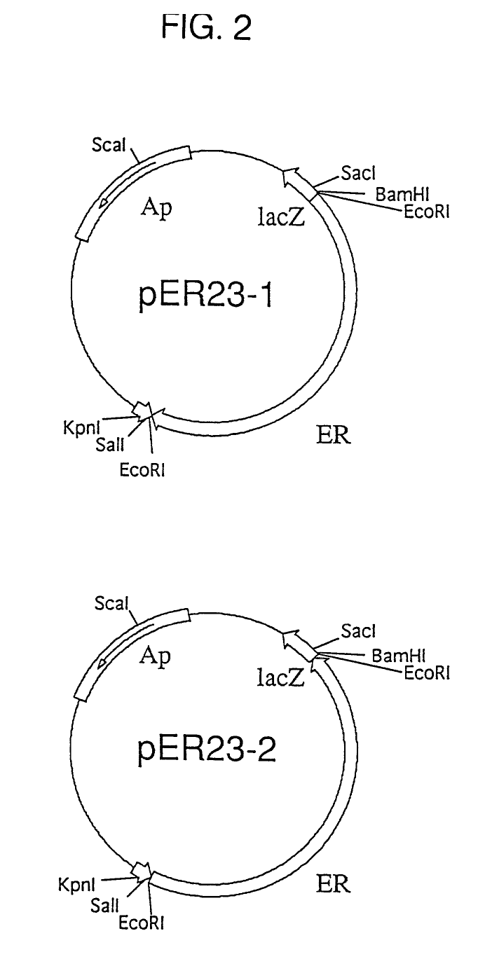

After the two DNA solutions were mixed, 2 μ1 of a 10 ×ligase buffer and 0.2 μ1 of T4 DNA ligase (Takara Shuzo Co., Ltd.) were added to give a total amount of 20 μ1. The mixture was reacted at 16° C. for 4 hours and the reaction mixture solution was used to transform E. coli DH5 α (Gibco BRL Co.). A 2% agar plate medium was prepared with 25 ml of an L medium containing 50 μg/ ml ampicillin, 40 μg/ ml IPTO and 40 μg/ml X-gal. The transformed E. coli was inoculated on the agar plate medium and grown at 37° C. overnight. White colonies were selected from the formed colonies and cultured in 3 ml of an L medium Containing 50 μg/ ml ampicillin at 37 ° C. for 8 hours. Plasmids were recovered from these bacterial cells by an alkaline method and determined whether they were clones into which a desired fragment was cloned with the restriction enzyme, thereby giving plasmids pER23-1 and pER23-2 (5225 bp) which had opposite orientations to the vector. The maps of plasmids pER23-1 and pER23-2 are shown in FIG. 2.

5) Determination of the Nucleotide Sequence of the ER-encoding Clone

The DNA nucleotide sequences of plasmids pER23-1 and pER23-2 were determined in both orientations with a fluorescence sequencer by 1) using plasmids pER23-1 and pER23-2 digested by appropriate restriction enzymes, 2) using appropriate primers synthesized on the basis of the information about already determined nucleotide sequences, or 3) cleaving pER23-1 with restriction enzymes Kpnl and Xhol and pER23-2 with restriction enzymes Kpnl and Cial and then using a kilosequence deletion kit (Takara Shuzo Co., Ltd.) to prepare plasmids having a deletion at intervals of about 200-300 bp. The DNA nucleotide sequence is shown in SEQ ID NO: 2 of the SEQUENCE LISTING. The results showed that the DNA fragment contained a 667 amino acid-encoding open reading frame of 2001 bp beginning at a nucleotide sequence corresponding to the N-terminal sequence (fragmented peptide #1) sequenced with the amino acid sequencer. The amino acid sequence is shown in SEQ ID NO: 1 of the SEQUENCE LISTING. The amino acid sequence deduced from the resulting DNA nucleotide sequence was consistent with the previously determined amino acid sequence of the soybean ER.

In addition, highly homologous amino acid sequences were searched for with a nucleic acid and amino acid sequence analysis software package (MacVector: Kodak Co.) using a nucleic acid and amino acid sequence data base (Entrez: NCBl). However, no amino acid sequences were found to be highly homologous to the heretofore known sequences. Hence, it is clear that the prepared ER is a novel protein.

### EXAMPLE 3

Expression of the Soybean ER in Tobacco Plants

1) Construction of Plant Expression Plasmid pKV1-ER23

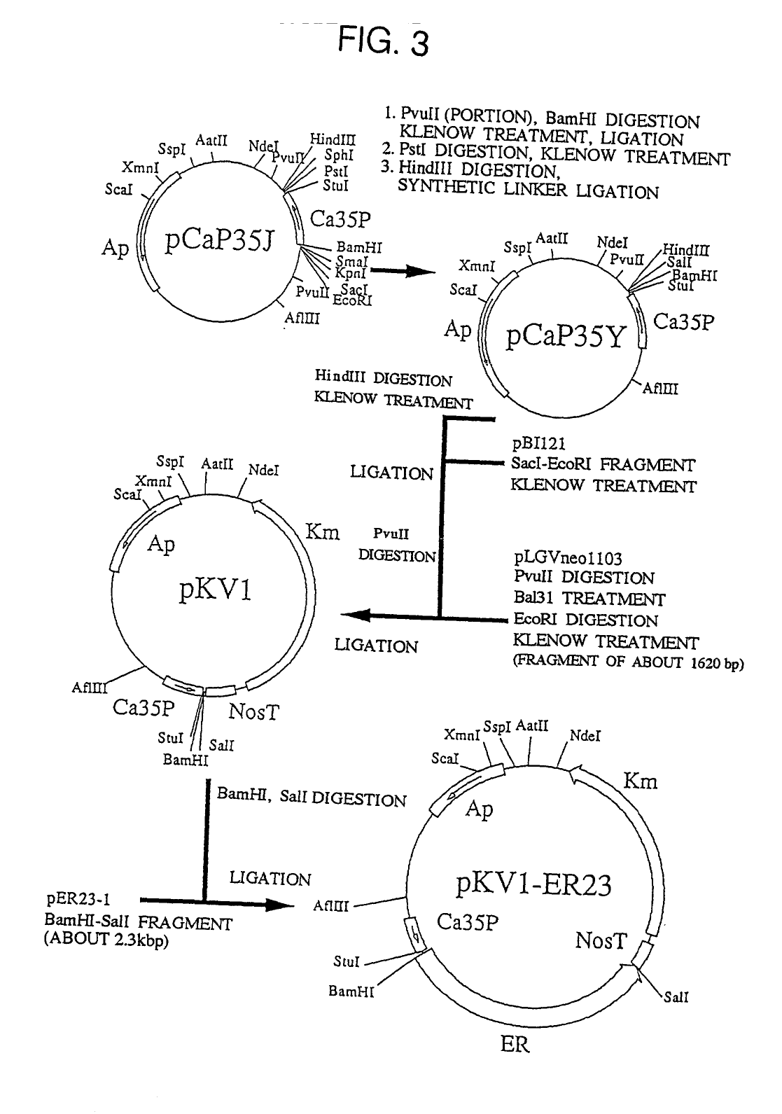

As shown in FIG. 3, a plant expression vector pKV1 to be used in this example was prepared from cauliflower mosaic virus 35S promoter-containing plasmid pCaP35J (J. Yamaya et al. (1988) Mol. Gen. Genet. 211:520) as follows:

Plasmid pCaP35J was digested completely with restriction enzyme BamHl to delete a multi-cloning site present upstream of the 35S promoter. Following partial digestion with Pvull, a treatment was conducted with Klenow fragments (Takara Shuzo Co., Ltd.) to make blunt ends. The resulting plasmid DNA was circularized by ligation and introduced into E. coli DH5 α. A desired plasmid was selected from the resulting clones. The selected plasmid was digested with restriction enzyme Pstl to insert a multi-cloning site present downstream of the 35S promoter. A treatment was conducted with Klenow fragments to make blunt ends. The resulting plasmid DNA was digested with Hindlll. The following synthetic linker DNAs were synthesized with an automatic nucleic acid synthesizer, annealed and ligated to the Hindlll-digested plasmid. The resulting plasmid DNA was introduced into E. coli DHS α Desired plasmid pCaP35Y (2837 bp) was selected from the obtained clones.

5,-GGAATTCGAGCTCGGTACCCGGGGGATCCTCT AGAGTCGACCTGCAGGC ATGCA-3′ (SEQ ID NO:15)

5′-CCTTAAGCTCGAGCCATGGGCCCCCTAGGAGATCTCAGCTGGACGTCCG TACGTTCGA-3′ (SEQ ID NO:16)

In order to introduce a terminator of nopaline synthase into the pCaP35Y, plasmid pBl121 (Clontech Co.) was digested with Sacl and EcoRl and the Saci-EcoRl fragment was treated with Klenow fragments to make blunt ends; then, the resulting fragment of pBl121 was ligated to plasmid pCaP35Y in which blunt ends were made at a Hindlll site downstream of the 35S promoter. The resulting plasmid DNA was introduced into E. coli DH5 α. A desired plasmid was selected from the obtained clones. In order to introduce a kanamycin-resistance cassette into the selected plasmid, the latter was digested with Pvull and ligated to a fragment (about 1620 bp) of pLGVneol 1103 (R. Hain et al. (1985) Mol. Gen. Genet. 199: 161) that was obtained by the steps of cleavage at a Pvnll site present downstream of the octopine synthase terminator, treatment with Ba131 (Takara Shuzo Co., Ltd.) to make a deletion, cleavage at a EcoRl site upstream of a nopaline synthase promoter, and the creation of a blunt end at both ends. The resulting plasmid DNA was introduced into E. coli DH5 α. Desired plasmid, or plant expression vector pKV1 (4828 bp), was selected from the obtained clones.

The prepared pKVl was digested at unique sites by restriction enzymes BamHl and Sall and ligated to the ER gene-containing fragment (i.e., the BamHl-Sall fragment of pEB23-1, about 2.3 kbp). The resulting plasmid DNA was introduced into E. coli DHS α. Desired ER-expression plasmid pKV1-ER23 (about 7.1 kbp) was selected from the obtained clones.

2) Transient Expression of ER in Cultured Tobacco Cells

The ER gene was introduced into cultured tobacco cells by electroporation for transient ER expression by a partial modification of Watanabe's method (Y. Watanabe (1987) FEBS 219: 65). The DNA molecules of the plasmid pKV1-ER23 were purified by an alkaline method. Cultured tobacco cells were obtained by the method of Hirai et al. (Plant Cell Cultivation Manual, Gakkai Shuppan Center, 1982) for use in the transient expression of ER. Tobacco seeds (variety Bright Yellow, provided by Professor Hirofumi Uchimiya of University of Tokyo) were sterilized with a 1% sodium hypochlorite solution and then germinated. The tobacco juvenile tissues just after the germination were transplanted in a tobacco cultivation agar medium (Murashige-Skoog medium (Flow Laboratories Co.) supplemented with 2 ppm 2,4-dichlorophenoxyacetic acid, 3% sucrose and 8% agar) to induce calli after 3 weeks. About 1 g of callus masses were suspended in 50 ml of a tobacco cultivation medium (Murashige-Skoog medium (Flow Laboratories Co.) supplemented with 2 ppm 2,4-dichlorophenoxyacetic acid and 3% sucrose) to prepare cultured cells. These tobacco cells were cultured until they entered a logarithmic growth phase. The cultured cells were collected by centrifugation (600 rpm, 3 minutes) and suspended in a solution consisting of 1% cellulase Onozuka (Yakult Co.), 1% Dricelase (Kyowa Hakko Co., Ltd.), 0.1% Pectriase (Seishin Seiyaku Co.) and 0.4 M D-mannitol (Wako Pure Chemicals Co., Ltd.) and which was adjusted to pH 5.7 with HCl. Reaction was performed at 30° C. for 90 minutes to prepare protoplasts. The reaction solution was washed with 0.4 M D-mannitol at 4° C. by 3 cycles of centrifugation to remove the enzyme solution. The operation of electroporation consisted of suspending 1×10⁶ cells in 0.8 ml of an electroporation solution (70 mM KCl, 5 mM MES and 0.3 M mannitol), mixing the suspension with 10 μg of the DNA molecules of pKVI-ER23 and treating the mixture with a Gene Pulser (Biorad Co.) at 125 μ F and 300 V in an electroporation cuvette (Biorad Co., electrode spacing: 0.4 cm). After the treatment, the solution was collected with a pasteur pipet and left to stand on ice for 30 minutes. Reaction was performed at 30° C. for 5 minutes and the reaction solution was resuspended in a protoplast medium (Murashige-Skoog medium (Flow Laboratories Co.) supplemented with 0.2 ppm 2,4-dichlorophenoxyacctic acid, 1% sucrose and 0.4 M mannitol and adjusted to pH1 5,7). The cells were left to stand in the dark at 25° C. overnight and collected by centrifugation (8,000 rpm, 3 minutes). Sixty microliters of a suspension buffer (25 mM Tris-HC1 pH7.0, 30 mM MgC1_{2,}2 mM dithiothreitol, 2,5 mM potassium metabisulfite and 1 mM PMSF) were added to the cells and the mixture was stirred on a vortex for 3 minutes. The resulting sample was stored at −80° C. until an elicitorbinding experiment was conducted.

For control, the above procedure was repeated except that the DNA molecule of pKV1 instead of pKV1-ER23 was introduced into tobacco cells.

3) Stable Expression of ER in Tobacco Suspension Cultured Cells

Transformed cultured tobacco cells capable of constant ER gene retention were selected as follows from the cultured tobacco cells capable of transient ER expression:

The protoplasts obtained in the preparation of the cultured tobacco cells capable of transient ER expression were suspended in a 1% agarose containing protoplast medium (Murashige-Skoog medium (Flow Laboratories Co.) supplemented with 0.2 ppm 2,4-dichlorophenoxyacetic acid, 1% sucrose and 0.4 M mannitol and adjusted to pH 5.7). The suspension was dropped on a plate with a dropping pipet before the agarose was solidified, whereby the protoplasts were fixed in the bead-like solid medium. After the agarose was solidified, an agarose-free protoplast medium was added to the plate, thereby immersing the protoplast-fixing agarose medium in the liquid medium. After the protoplasts were cultured in the dark for 1 week, kanamycin was added to a final concentration of 100 μ/ml and the cultivation was continued. Transformants selected from the grown colonies were transferred in a kanamycin-containing liquid medium and cultured.

Two clones (|1 and |6) of cultured tobacco cells stably transformed by pKV1-ER23 and two clones (C 2-1 and C 2-4) of cultured tobacco cells stably transformed by pKV| were obtained.

4) Elicitor-binding Activity Experiment

The elicitor-binding activity was measured as follows:

A complex of an elicitor and tyramine (Tokyo Kasei Kogyo Co., Ltd.) was synthesized by the method of Jong-Joo Cheong (The Plant Cell (1991) 3:127). The elicitor-tyramine complex was labelled with iodine-125 using chloramine T. The resulting sample (protein amount <500 μg) was suspended in 500 μ1 of an assay buffer (50 mM Tris-HC1 pH7.4, 0.1 M saccharose, 5 mM MgC12, 1mM PMSF and 5 mM EDTA) and incubated at 0° C. for 2 hours. The iodine-labelled elicitor-tyramine complex in an amount of 100 nM (70 Ci/mmol) was added to the suspension and the mixture was incubated at 4° C. for 2 hours. The reaction solution was filtered through Whatman GF/B (as treated with a 0.3% aqueous solution of polyethylenimine for at least 1 hour) and washed 3 times with 5 ml of an ice-cold buffer (10 mM Tris-HCl pH 7.0, 1 M NaCl, 10 mM MgCl₂). The radioactivity retained on the filter membrane was counted with a gamma counter (count A). In order to eliminate the effect of non-specific binding, the same procedure as above was performed except that 17 μ M of the elicitor was added to the same sample, the mixture was suspended in the assay buffer, and the suspension was incubated at 0° C. for 2 hours. The obtained count was subtracted from the count A to give a count (Δ cpm) of elicitor-specific binding. The resulting count (Δ cpm) was divided by the total number of counts and then multiplied by the total amount of elicitor used in the experiment to calculate the amount of the elicitor-binding protein (in moles).

As a result, a specific binding to the elicitor was observed in the tobacco cells transformed with the DNA molecule of pKVl-ER23, whereas no specific binding to the elicitor was observed in the control tobacco cells in which the DNA molecule of pKVl was introduced (Table 2). This fact reveals that the gene obtained above encodes a protein having the elicitor-binding activity.

TABLE 2
Elicitor-binding Activity of Cultured Tobacco Cells

| Fraction | Transforming DNA | Binding Activity (fmol/mg) |
|---|---|---|
| Transient Expression | pKV1 | <0 |
| | pKV1-ER23 | 90.5 |
| Stable Expression | | |
| C2-1 | pKV1 | <0 |
| C2-4 | PKV1 | <0 |
| I1 | pKV1-ER23 | 150 |
| I6 | pKV1-ER23 | 196 |

5) Transient Increase in Intracellular Ca²⁺Concentration in Transformed Tobacco Cultured Cells by Addition of Glucan Elicitor.

Plants recognize the elicitor by a specific receptor thereto and them promote the accumulation of phytoalexin or induce hypersensitive reaction to prevent fungus invasion. It has been reported for some plants that the inflow of calcium ion into cells in the early phase of such resistance reactions is important (U. Conrath et al. (1991) FEBS LETTERS 279: 141, M. N. Zook et al. (1987 )Plant Physiol. 84: 520, F. Kurosaki et al. (1987 )Phytochemistry 26: 1919; C. L. Preisig and R. A. Moreau (1994)Phytochemistry 36: 857). A report has also been made suggesting that the inflow of calcium ion into cells triggers the promotion of the phytoalexin accumulation in soybean, which the present inventors used to obtain ER (M. R. Stab and J. Ebel (1987) Archi. Biochem. Biophys. 257: 416). Hence, if a transformed cultured tobacco cell is prepared by introducing the ER gene into an ER-free tobacco cultured cell to express the ER and if the intracellular calcium ion concentration is changed by the addition of a glucan elicitor, the change is anticipated to trigger a resistance reaction by the glucan elicitor in plants other than soybean (e.g., tobacco), thereby allowing them to show resistance to a wide variety of fungi which use glucan as a mycelial wall component.

The change in intracellular Ca²⁺concentration of transformed cultured tobacco cells by the addition of the elicitor was examined.

In this experiment, the transformed cultured tobacco cells (|6) obtained by the kanamycin selection and the plasmid-containing cultured tobacco cells (C 2-4) were used.

The intracellular Ca²⁺concentrations of the cultured cells were measured as follows with an acetoxynmethyl derivative (Fura-2 AM) of a fluorescence chelator (Fura-2) for Ca²⁺measurement:

Cells were harvested from about 2 ml of the transformed tobacco cell culture (corresponding to a cell volume of about 250 μ1 after standing for 10 minutes) by centrifugation (600 rpm, 30 seconds) and the supernatant was removed. To the cells was added 2 ml of a tobacco cultivation medium and the mixture was stirred gently and centrifuged (600 rpm, 30 seconds) to remove the supernatant. The same operations were repeated to wash the cultured cells. The washed cultured cells was suspended homogeneously in 2 ml of the medium. To 1 ml of the suspension of the cultured cells in the medium, 1 ml of the medium and 4 μ1 of 1 mM Fura-2 AM (final concentration: 2 μM, Dojin Chemical Co.) were added and the mixture was incubated in the dark for 30 minutes with occasionally stirring. Subsequently, the cells were washed 2 times with 2 ml of the medium by centrifugation (600 rpm, 30 seconds) to eliminate the free Fura-2 AM which was not incorporated into the cells. The washed cultured cells were suspended in 2 ml of the medium homogeneously and the suspension (2 ml) was transferred into a fluorescence-measurement cell. The incorporated Fura-2 AM should be changed to Fura-2 by hydrolysis with intracellular esterase. The fluorescence produced by the binding of Fura-2 to intracellular Ca²⁺was measured at a fluorescence wavelength of 505 nm under exciting light of 335 nm with the cultured cells being stirred to ensure against precipitation of the cultured cells. The change in intracellular Ca²⁺concentration was examined by measuring the fluorescence intensity at specified intervals of time after the addition of 50 μ1 of glucan elicitor (1 mg/ml) or deionized water to the cultured cells. For control, the change in intracellular Ca²⁺concentration was examined on the plasmid-containing cultured tobacco cells by the same method as above.

For another control, the change in intracellular Ca²⁺concentration was examined on cultured soybean cells by the same method as above, except that the cultured cells were washed with a medium, for soybean cells having the following formulation, NaH₂PO₄H₄O 75mg/ml, KH₂PO₄170mg/ml, KNO₃2,200mg/ml, NH₄NO₃600mg/ml, (NH₄)₂SO₄ 67mg/ml, MgSO₄·7H₂O 310mg/ml, CaCl₂2H₂O 295mg/ml, FeSO₄ 7H₂O 28mg/ml, EDTA Na₂37.3mg/ml, Kl 0.75mg/ml, MnSO₄H₂O 10.0mg/ml, H₃BO₃3.0mg/ml, ZnSO₄7H₂O 2mg/ml, Na₂ MoO₄2H₂O 0.25mg/ml, CuSO₄5 H₂O 0.025mg/ml, COCl₂6H₂O 0.025mg/ml, Inositol 100mg/ml, Nicotinic acid 1.0mg/ml, Pyridoxine HCl 1.0mg/ml, Thiamine Hl1 10.0mg/ml, Glucose 5g/ml, Sucrose 25g/ml, Xylose 250mg/ml, Sodium pyruvate 5.0mg/ml, Citric acid 10.0mg/ml, Malic acid 10.0mg/ml, Fumaric acid 10.0mg/ml, N-Z-amine 500.0mg/ml, 2,4-dichlorophenoxyacetic acid 1.0mg/ml and Zeatine riboside 0.1 mg/ml, adjusted to pH 5.7 with KOH.

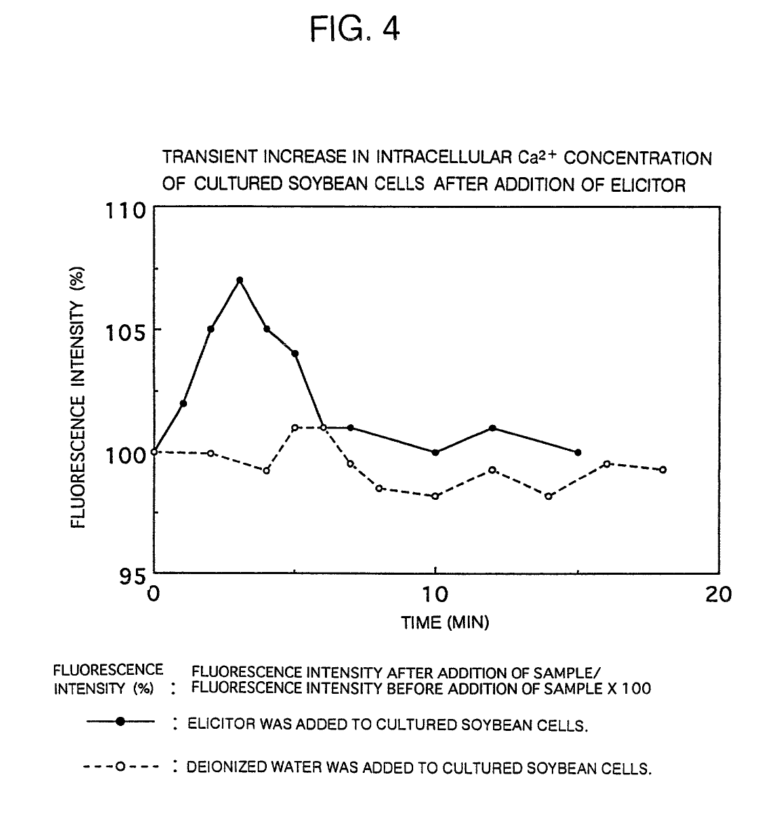

As a result of this experiment, about 7% transient increase in fluorescence intensity was observed in the cultured soybean cells 3 minutes after the addition of the elicitor, whereas no such change was observed after the addition of deionized water (FIG. 4). The results suggest that the phenomenon in which the binding of the ER to the glucan elicitor caused a transient inflow of Ca²⁺into cells could be observed in this experiment, thereby supporting the report that calcium ion plays an important role in the resistance reaction caused by the elicitor in cultured soybean cells in the transformed cultured tobacco cells, about 10% transient increase in fluorescence intensity was observed 3 minutes after the addition of the elicitor, whereas no such change was observed after the addition of deionized water.

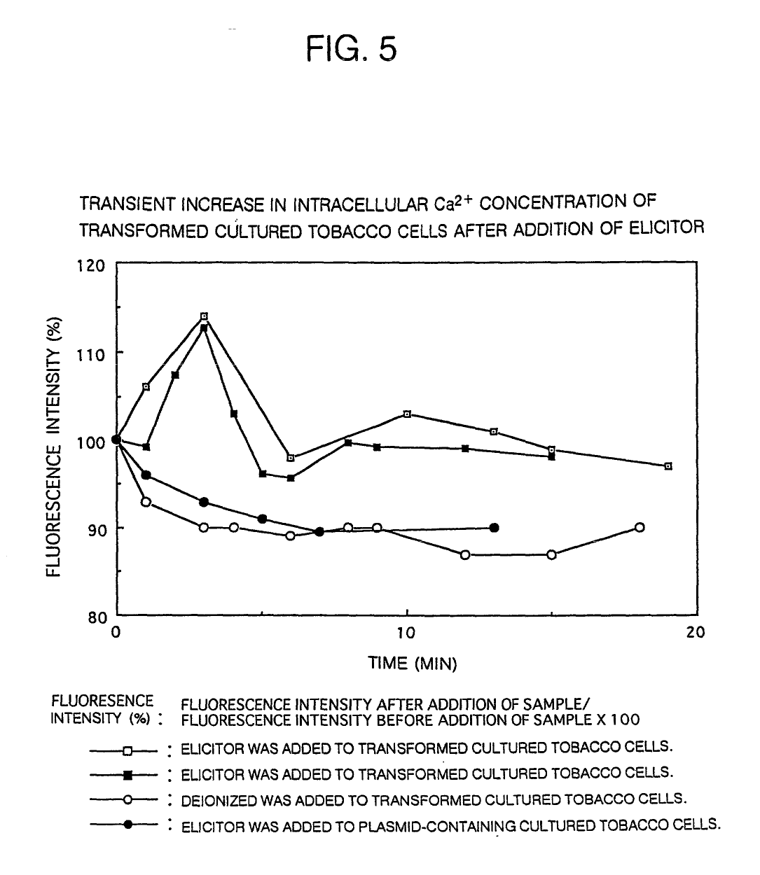

In the plasmid-containing cultured tobacco cells, none of the changes in fluorescence intensity that occurred in the transformed cultured tobacco cells was observed after the addition of the elicitor (FIG. 5).

These results show that plants other than soybean (e.g., tobacco), which are not reactive with the glucan elicitor acquire the reactivity by introducing the gene of the soybean-derived glucan elicitor receptor for ER expression. Although the signal transduction pathway of each plant has not been completely explicated, it is expected that plants other than tobacco will acquire the reactivity with the glucan elicitor (i.e., a transient increase in intracellular Ca²⁺concentration) by introducing the gene of the present ER for ER expression, thereby enabling the development of plants having resistance to a wide variety of fungi which use glucan as a mycelial wall component.

### EXAMPLE 4

Expression of Soybean ER in E. coli and Determination of Elicitor-binding Domain

1) Expression of Elicitor-binding Domain in E. coli.

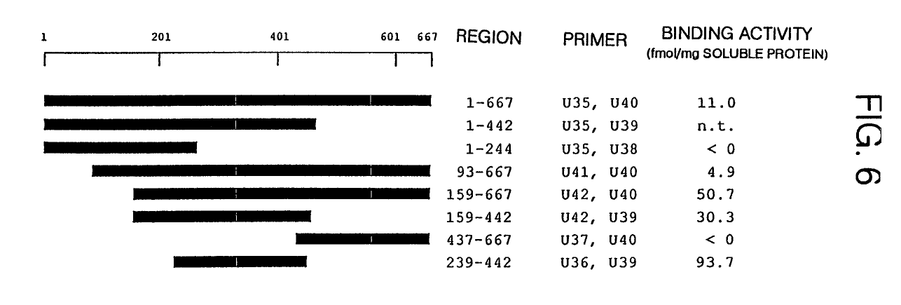

A fused protein of a partial fragment of the soybean ER with a maltose-binding protein (MBP) was prepared with a Protein Fusion & Purification System (New England Biolabs Co.) in order to express the partial fragment of the soybean ER in E. coli. PCR was performed using pERZ3-1 as a template to give DNA fragments of various lengths. The primers were designed to produce the MBP and fused protein in cloning into plasmid pMAL-c2 (New England Biolabs Co.) by adding a BamHl site on the 5′ side and a Sall site on the 3′ side exterior to the DNA molecule encoding the full-length portion and fragments of soybean ER shown in FIG. 6.

These primers were synthesized with an automatic nucleic acid synthesizer (Model 394 of Applied Biosystems Co.). The following primers were used in the amplification of the DNA chain.

Primer U35 5′-ATGGATCCATGGTTAACAT CCAAACC-3′(SEQ ID NO:17);

Primer U36 5′-ATGGATCCGAATATAACT GGGAGAAG-3′(SEQ ID NO:18);

Primer 1137 5′-ATGGATCCCCAGCAT GGGGTAGGAAG-3′(SEQ ID NO:19);

Primer 1138 5′-TAGTCGACTACTTCTCCCA GTTATATTC-3′(SEQ ID NO:20);

Primer U39 5′-TAGTCGACTACTTCCTACCCC ATGCTGG-3′(SEQ ID NO:21);

Primer U40 5′-TAGTCGACTATTCATCACTTC TGCTATG-3′(SEQ ID NO:22);

Primer U41 5′-ATGGATCCGCCCCACAA GGTCCCAAA-3′(SEQ ID NO:23);

and

Primer 1142 5′-ATGGATCCAATGACTCCAA CACCAAG-3′(SEQ ID NO:24)

The DNA molecule of pER23-l (0.01 μg) was dissolved in 79 μ1 of distilled water. Either a combination of primers U5 and μ or a combination of primers μ 10 and U12 (100 pmol each) and 0.5 μ1 of Taq DNA polymerase (Takara Shuzo Co., Ltd.) were added to 8 μ1 of 2.5 mM dNTPs in 10μl of a 10×PCR buffer (attached to Taq DNA polymerase of Takara Shuzo Co., Ltd.) to give a final amount of 100 μ1. PCR reaction was performed with a Gene Amp PCR System 9600 (Perkin-Elmer Co.) by 30 cycles of 1) denaturation at 94° C. ×30 seconds, 2) renaturation at 55° C. ×30 seconds and 3) extension at 72° C. ×1 minute. After the reaction, 15 μ1 of the reaction solution was digested with restriction enzymes BamHl and Sall and electrophoresed on a 1% agarose gel.

The gel was stained with a 0.5 μg/ml ethidium bromide solution for 15 minutes. The band showing the expected specific amplification was sectioned while observing under UV light. The gel section was treated with Gene Clean II (Bio101Co.) to collect a DNA-containing solution. The collected DNA fragments were cloned into the BamHl-Sall site of plasmid pMAL-c2 and the clones were introduced into E. coli DH5 α.

2) Preparation of Soluble Protein Fraction from E. coli.

The E. coli cells into which the plasmids were introduced were precultured in an expression medium [10g/l tryptone (Gibco Co.), 5 g/1 yeast extract (Gibco Co.), 5 g/l NaCl, 2 g/l glucose and 100 μg/ml ampicillin]. The precultured solution (0.4 ml) was added to 40 ml of the expression medium and cultured at 37° C. with shaking until OD₆₀₀ of 0.55 was reached. Isopropylthiogalactoside was added to the culture solution to give a final concentration of 0.3 mM and the shaking culture was continued for an additional 4 hours to induce expression. The E. coli was collected by centrifugation and the E. Coli cells were washed with a washing buffer (20 mM Tris-HC1, pH 7.4, 200 mM NaCl and 1 mM EDTA). The cells were sonicated for a total of 2 minutes (15 sec ×8). ZW3-12 was added to the sonicated cells to give a final concentration of 0.25% and the mixture was incubated at 4° C. for 30 minutes. The supernatant was collected by centrifugation (10,000 rpm, 5 minutes) to give an E. coli soluble protein fraction. The expression of the fused protein was confirmed by an immunoblotting technique using an antimaltose-binding protein antibody (New England Biolabs Co.).

3) Elicitor-binding Experiment

The elicitor-binding activity was determined as follows:

A complex of an elicitor and tyramine (Tokyo Kasei Kogyo Co., Ltd.) was synthesized by the method of Jong-Joo Cheong (The Plant Cell (1991) 3: 27). The elicitor-tyramine complex was labelled with iodine-125 using chloramine T. The resulting sample (protein amount <800μ g) was suspended in 500 μ1 of an assay buffer (50 mM Tris-HCl pH7.4, 0.1 M-saccharose, 5 mM MgC1_{2,}1mM PMSF and 5 mM EDTA) and incubated at 0° C. for 2 hours. The iodine-labelled elicitor-tyramine complex in an amount of 100 nM (70 Ci/mmol) was added to the suspension and the mixture was incubated at 4° C. for 2 hours. The reaction solution was filtered through Whatman OF/B (as treated with a 0.3% aqueous solution of polycthylenimine for at least 1 hour) and washed 3 times with 5 ml of an ice-cold buffer (10 mM Tris-HC1 pH 7.0, 1 M NaCl, 10 mM MgCl2). The radio activity retained on the filter membrane was counted with a gamma counter (count A). In order to eliminate the effect of non-specific binding, the same procedure as above was performed except that 17 μM of the elicitor was added to the same sample, the mixture was suspended in the assay buffer and the suspension was incubated at 0° C. for 2 hours. The obtained count was subtracted from the count A to give a count (Δ cpm) of elicitor-specific binding. The resulting count (Δ cpm) was divided by the total number of counts and then multiplied by the total amount of the elicitor used in the experiment to calculate the amount of the elicitor-binding protein (in moles).

As a result, a specific binding to the elicitor was observed in the E. coli transformed with the DNA molecule encoding the ER (FIG. 6). Hence, it was reconfirmed that the obtained gene encoded a protein having the elicitor-binding activity and it was revealed that there was an elicitor-binding domain in the 239-442 amino acid sequence of SEQ ID NO:1.

### EXAMPLE 5

Inhibition of Binding of Glucan Elicitor to Elicitor-binding Protein in Soybean Cotyledon Membrane Fraction and Inhibition of Accumulation of Phytoalexin in Soybean Cotyledon by Antibody against Elicitor-binding.

Domain

1) Expression of Elicitor-binding Domain in E. coli

A fused protein of an elicitor-binding domain derived from the ER with a maltose-binding protein (MBP) was prepared with a Protein Fusion & Purification System (New England Biolabs Co.) in order to express a large amount of the elicitor-binding domain in E. coli PCR was performed to produce a DNA molecule encoding the elicitor-binding domain. The following primers were synthesized with an automatic nucleic acid synthesizer (Model 394 of Applied Biosystems Co.):

Primer 1.136 5′ATGGATCCCfAATATAACT CIGGAGAAG 3′(SEQ ID NO:25); and

Primer U39 5′-TAGTCGACTACTTCCTACCC CATc3CTGG-3′(SEQ ID NO:26)

The DNA molecule of pER23-l (0.01 μg) was dissolved in 79 μ1 of distilled water. Either a combination of primers U5 and U7 or a combination of primers U10 and U12 (100 pmol each) and 0.5μ1 of Taq DNA polymerase (Takara Shuzo Co., Ltd.) were added to 8 μ1 of 2.5 mM dNTP in 10 μ1 of a 10×PCR buffer (attached to taq DNA polymerase of Takara Shuzo Co., Ltd.) to give a final amount of 100 μ1. PCR was performed with a Gene Amp PCR System 9600 (Perkin-Elmer Co.) by 30 cycles of 1) denaturation at 94° C. ×30 seconds, 2) annealing at 55° C. ×30 seconds and 3) extension at 72° C. ×1 minute. After the reaction, 15μ1 of the reaction solution was digested with restriction enzymes BamHl and Sall and electrophoresed on a 1% agarose gel.

The gel was stained with a 0.5 μg/ml ethidium bromide solution for 15 minutes. The band showing specific amplification was sectioned while observing under UV light. The gel section was treated with Gene Clean II (Bio101Co.) to collect a DNA-containing solution. The collected DNA fragments were cloned into the BamHl-Sall site of plasmid pMAL-c2 and the clones were introduced into E. coli DH5

2) Purification of the Fused Protein Expressed in E. coli and Production of Antibody.

The E. coli cells transformed with the plasmids were precultured in an expression medium (10g/l tryptone (Gibco), 5 g/l yeast extract (Gibco), 5 g/l NaCl 2 g/l glucose and 100 μg/ml ampicillin) overnight. The precultured solution (150 ml) was added to 1.5 L of the expression medium and cultured in a Sakaguchi flask at 37° C. with shaking until OD₆₀₀ of 0.55 was reached. lsopropylthiogalactoside was added to the culture solution to give a final concentration of 0.3 mM and the shake culture was continued for an additional 4 hours to induce expression. The E. coli was collected by centrifugation and the E. coli cells were washed with a washing buffer (20 mM Tris-HCl, pH 7.4, 200 mM NaCl and 1 mM EDTA). The cells were sonicated for a total of 2 minutes (15 sec ×8). A soluble protein fraction was obtained by centrifugation. From this fraction, a MBP-fused protein was purified with an amylose resin. A MBP-and an elicitor-binding domain were cleaved with factor Xa and the elicitor-binding domain was purified by gel filtration column chromatography. The purified protein was injected twice into a mouse at the abdominal cavity for immunization by the method of E. Harlow and D. Lane (Antibody (1988) Cold Spring Harbor Co., pp. 53-137). After the increase in titer was confirmed by an ELISA method, the ascites was obtained and subjected to precipitation with 50% saturated ammonium sulfate and treated with Protein A Sepharose (Pharmacia Co.) to produce a purified antibody. In the treatment with Protein A Sepharose, the antibody was bound to Protein A Sepharose with 0.1 M sodium phosphate (pH 8.0) and eluted with 0.1 M citric acid (pH 3.5). It was confirmed by an immunoblotting that the obtained antibody recognized only the ER protein in soybean.

3) Preparation of Soybean Cotyledon Membrane Fraction.

A soybean cotyledon membrane fraction was prepared as follows:

To soybean cotyledons cultured on soil for 9 days (wet weight: 36 g), 47 ml of an ice-cooled buffer (25 mM Tris-HCl, pH 7.0, 30 mM MgCl2, 2 mM dithiothreitol, 2.5 mM sodium metabisulfite, 1 mM PMSF) was added and homogenized with a waring blender, followed by fractionation through centrifugation to form a precipitate of the cotyledon membrane fraction; the procedure was the same as in the preparation of the soybean root membrane fraction described in Section 2) of Example 1. The cotyledon membrane fraction described in Section 2) of Example 1. The cotyledon membrane fraction was suspended in an ice-cooled buffer (10 mM Tris-HCl, pH 7.4, 0.1 M sucrose, 5 mM MgC_{2,} 1 mM PMSF, 5 mM EDTA) and stored at −80° C.

4) Measurement of Inhibition of Glucan Elicitor Binding to Elicitor-binding Protein of Soybean Cotyledon Membrane Fraction.

The elicitor-binding activity was determined as follows:

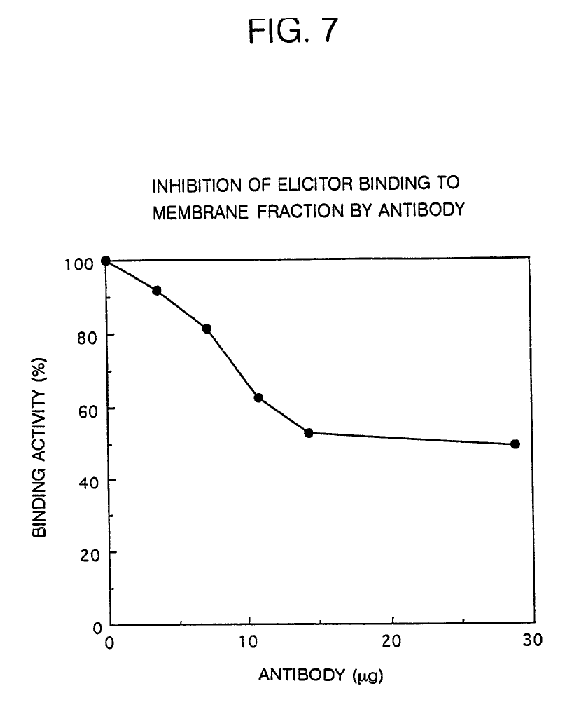

A complex of an elicitor and tyramine (Tokyo Kasei Kogyo Co., Ltd.) was synthesized by the method of Jong-Joo Cheong (The Plant Cell (1991) 3:127). The elicitor-tyramine complex was labelled with iodine-125 using chloramine T. The soybean cotyledon membrane fraction (100 μ1, 820 μg) was suspended in 500 μ1 of an assay buffer (50 mM Tris-HC pH7.4, 0.1 M saccharose, 5 mM MgCl_{2,}1mM PMSF and 5 mM EDTA) and incubated at 0° C. for 2 hours. The iodine-labelled elicitor-tyramine complex in an amount of 714 ng (143 nM; 70 Ci/mmol) was added to the suspension and the mixture was incubated at 4° C. for 2 hours. The reaction solution was filtered through Whatman GF/B (as treated with a 0.3% aqueous solution of polyethylenimine for at least 1 hour) and washed 3 times with 5 ml of an ice-cold buffer (10 mM Tris-HCl pH 7.0, 1 M NaCl, 10 mM MgCl2). The radio activity retained on the filter membrane was counted with a gamma counter (count A). In order to eliminate the effect of non-specific binding, the same procedure as above was performed, except that 100 times mole (75 μg, 15 μM) of a cold elicitor was added to the sample, the mixture was suspended in the assay buffer and the suspension was incubated at 0° C. for 2 hours. The obtained count was subtracted from the count A to give a count (Δ cpm) of elicitor-specific binding. Counts of binding obtained by adding 3.6, 7.1, 10.8, 14.4 and 28.8 μg of the purified antibody rather than the cold elicitor were subtracted from the count A. The resulting values were compared with that for the cold elicitor and expressed as the percentage, with the count (Δ cpm) of elicitor-specific binding being taken as 100% (FIG. 7). The addition of 28.8 μg of the antibody resulted in the inhibition of the binding of elicitor by about 51%. The results confirmed that the antibody against the elicitor-binding domain inhibited the binding of the elicitor to the elicitor-binding protein.

5) Inhibition of Accumulation of Phytoalexin by Antibody against Elicitor-binding Domain.

The amount of phytoalexin accumulated by the action of glucan elicitor was measured with soybean cotyledons by the method of M. G. Hahn et al. ((1992) Molecular Plan Pathology Volume II A Practical Approach, lRL Press, pp. 117-120).

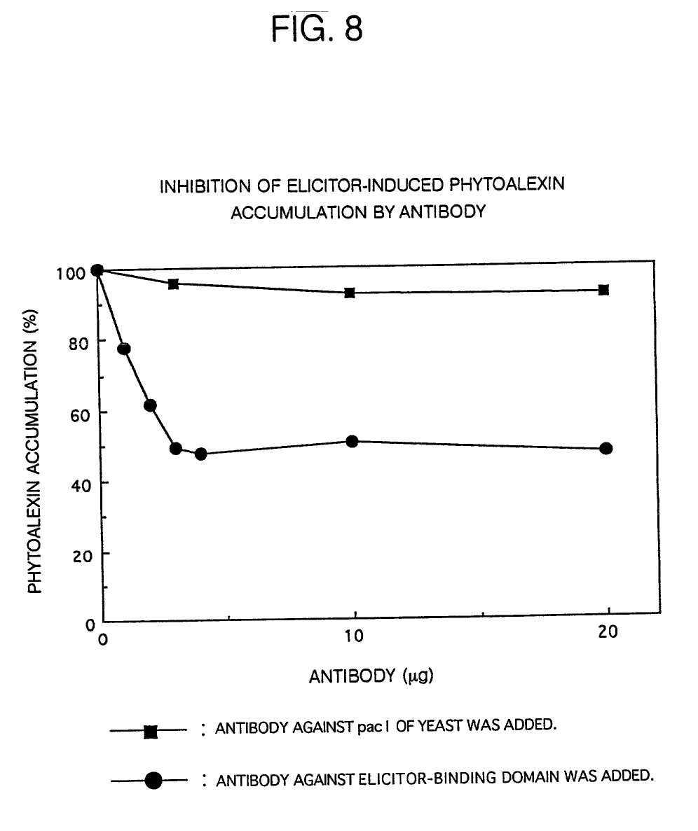

A purified antibody against the elicitor-binding domain (0, 1, 2, 3, 4, 10 and 20) μg/25 μ1/cotyledon) or a purified antibody against yeast-derived dsRNAse, pac 1 (4, 10 and 20 μg/25 μ1 /cotyledon) as a control was added to soybean cotyledons and the mixture was incubated for 1 hour. Glucan elicitor (200 ng/25) μ1 /cotyledon) was added to the soybean cotyledons and the mixture was incubated for 20 hours to determine whether the accumulation of phytoalexin by the action of glucan elicitor was inhibited by the antibody. The amount of phytoalexin accumulation induced by the addition of elicitor subsequent to the addition of the antibody was expressed as the percentage, with the amount of phytoalexin accumulation by the sole addition of the elicitor being taken as 100% (FIG. 8). When the antibody against the elicitor-binding domain was added in an amount of 20.0 μg per soybean cotyledon, the amount of phytoalexin accumulation decreased by about 53%. In the control, the amount of phytoalexin accumulation changed little even when the antibody against pac 1 was added in an amount of 20.0 μg per soybean cotyledon. These results showed that the obtained gene did not encode a mere elicitor-binding protein but encoded the ER inducing a resistance reaction in soybean.

### EXAMPLE 6

Transfer of ER Gene into Tobacco Plants

The soybean-derived ER gene was transferred into tobacco as described below, and the expression of the gene was confirmed.

1) Construction of Plant Expression Vector Plasmid

Plasmid pER23-1 is digested with BAMHl and Sall to give an ER gene fragment sandwiched between the sites of the two restriction enzymes. This fragment is inserted into a plant vector to be described below. In a separate step, a plant expression-type binary plasmid pBl121 (Clonetech) was digested with restriction enzymes BAMHl and Sacl. Then, the following linker DNAs synthesized with an automatic nucleic acid synthesizer were annealed and ligated to the digested binary plasmid, which was introduced into E. coli DH5 α. Desired plasmid pBllinker was selected from the obtained clones.

5′-CTAGAGGATCCGGTACCCCCGGGGTCGACGAGCT-3′(SEQ ID NO:27)

5′-CGTCGACCCCGGGGGTACCGGATCCT-3′(SEQ ID NO:28)

The gene fragment described above was inserted between the cauliflower mosaic virus 35S promoter and the terminator of nopaline synthase (BAMHl-Sall) in the resultant plasmid pBllinker to produce a vector to be introduced into plants (pBl-ER).

2) Introduction of pBl-ER into Agrobacterium

Agrobacterium tumefaciens LBA4404 (Clonetech) was inoculated into 50 ml of YEB medium (containing 5 g of beef extract, 1 g of yeast extract, 1 g of peptone, 5 g of sucrose and 2 mM MgSO₄ per liter, pH 7.4) and cultured at 28° C. for 24 hours. Then, the cells were harvested by centrifugation at 3,000 rpm and 4° C. for 20 minutes. The cells were washed three times with 10 ml of 1 mM Hepes-KOH (pH 7.4), once with 3 ml of 10% glycerol and finally suspended in 3 ml of 10% glycerol to give an Agrobacterium into which DNA of interest was to be introduced.

Fifty μ1 of the thus obtained bacterium suspension and 1 μg of plasmid pBl-ER were placed in a cuvette, to which an electrical pulse was applied at 25 μF, 2500 V and 200 Ω using an electroporation apparatus (Gene Pulser; BioRad) to introduce the plasmid DNA into the Agrobacterium. The resultant solution was transferred to an Eppendorf tube, followed by the addition of 800 μ1 of SOC medium (containing 20 g of tryptone, 5 g of yeast extract, 0.5 g of NaCl, 2.5 mM KC1, 10 mM MgSO_{4,}10 mM MgCl₂ and 20 mM glucose per liter, pH 7.0). The bacterium was subjected to stationary culture at 28° C. for 1.5 hours. Fifty μ1 of the resultant culture solution was plated on YEB agar medium (agar 1.2%) containing 1 00 ppm kanamycin and cultured at 28° C. for 2 days.

A single colony was selected from the resultant colonies, and plasmid DNA was prepared from that colony by an alkaline method. This plasmid DNA was digested with an appropriate restriction enzyme, and the resultant DNA fragments were fractionated by electrophoresis on 1% agarose gel and analyzed. As a result, it was confirmed that the plasmid DNA contained plasmid pBl-Er. This Agrobacterium tumefaciens is designated Agro-ER.

3) Transformation of Tobacco

The Agro-ER strain described above was cultured under shaking in LB liquid medium containing 50 ppm kanamycin at 28° C. for 2 hours. Cells were harvested by centrifuging 1.5 ml of the culture solution at 10,000 rpm for 3 minutes and washed with 1 ml of LB medium to remove the kanamycin. Then, the cells were harvested by further centrifugation at 10,000 rpm for 3 minutes and re-suspended in 1.5 ml of LB medium to give a bacterium suspension for infection.

In infecting a tobacco variety Bright Yellow with the bacterium, young leaves were collected from a germ-free plant. These leaves were aseptically cut into pieces 1 cm² in size with a surgical knife, placed on the Agrobacterium suspension with back of each leaf facing up, and shaken gently for 2 minutes. Thereafter, the leaf pieces were placed on a sterilized filter paper to remove excessive Agrobacterium. Whatman No. 1 filter paper (φ7.0 cm) was placed on MS-B5 medium (containing 1.0 ppm benzyladenine, 0.1 ppm naphthaleneacetic acid and 0.8% agar) (Murashige, T. and Skoog, F. Plant Physiol., 15: 473, (1962)) in a culture dish. The leaf pieces were placed upon this filter paper with the back of each leaf facing up. The culture dish was sealed with a PARAFILM (American National Can), and then the leaf pieces were cultured at 25° C. for 2 days through cycles of 16 hours under light and 8 hours in the dark. Subsequently, the leaf pieces were transferred onto MS-B5 medium containing 250 ppm claforan and cultured in the same manner for another 10 days in order to remove the Agrobacterium. The leaf pieces were further transferred onto MS-B5 medium containing 25 ppm claforan and 100 ppm kanamycin and cultured in the same manner for another 7 days. During this period, the regions surrounding the leaf pieces changed to callus, yielding shoot primordia. After culturing for another 10 days, elongated shoots were placed on MS-HF medium (benzyladenine-and napthaleneacetic acid-free MS-B5 medium) contain 250 ppm claforan and 100 ppm kanmycin. After culturing for 10 days, those shoots which were rooting were placed on MS-HF medium containing 250 ppm claforan in a plant box as kanamycin resistant transformant.

4) PCR and Immunoblot Analysis of Genomic DNA from the Transformant Tobacco

In order to confirm that the gene of interest was transferred into the transformant, a PCR was performed. The following primers were synthesized with an automatic nucleic acid synthesizer (Applied Biosystems; Model 394) and used in the PCR.

Primer ER1 5′-CACCTTCAGCAACAATGGTT-3′(SEQ ID NO: 29)

Primer ER2 5′-CTATTCATCACTTCTGCTAT-3′(SEQ ID NO:30)

DNA was extracted from the kanamycin resistant transformant tobacco and examined. Genomic DNA was extracted as described below. Briefly, 20 mg of tobacco leaves was crushed with a plastic bar in 200 μ1 of an extraction buffer (0.5 M NaCl, 50 mM Tris-HC1, pH 8, 50 mM EDTA). Then, 60 μ1 of 20% polyvinyl pyrrolidone (mean molecular weight: 40 kDA) and 52 μ1 of 10% SDS were added and heated at 65° C. for 30 minutes. Subsequently, 40 μ1 of 5 M potassium acetate was added, and the resultant mixture was left on ice for 30 minutes. Then, the mixture was centrifuged to recover the supernatant; 180 μ1 of isopropyl alcohol was then added to recover the DNA as a precipitate. After washing with 70% ethanol, the DNA was dissolved in 150 μ1 of TE solution (10 mM Tris-HC1, pH 8, 1 mM EDTA, 1 μg/ml RNAse A). To 70 μ1 of distilled water, 1 μ1 of this DNA solution, 10 μ1 of 10X PCR buffer (attachment to Taq DNA polymerase; Takara Shuzo, Co. Ltd.), 8 μ of 2.5 mM dNTPs, 100 pmol each of primers ER1 and ER2, and 0.5 μ1 of Taq DNA polymerase (Takara Shuzo Co., Ltd.) were added to make a 100 μ1 solution. With this solution, a PCR was performed as follows. As a reaction apparatus, Gene Amp PCR System 9600 (Perkin-Elmer) was used. First, denaturation reaction was performed at 94° C. for 5 minutes. Then, 30 cycles of 1) denaturation at 94° C. for 30 seconds, 2) annealing at 55° C. for 30 seconds and 3) extension at 72° C. for 1 minute were performed. After the reaction, 15 μ1 of the reaction solution was electrophoresed on 1% agarose gel. The gel was stained with a 0.5 μg/ml ethidium bromide solution for 15 minutes and examined under UV light. By confirming a specific DNA fragment of about 2 kbp which was expected to be amplified, it was confirmed that the gene of interest has been incorporated into the tobacco genomic DNA.

Immunoblot analysis was also performed to check for the expression of the gene of interest. Briefly, 20 mg of tobacco leaves was crushed with a plastic bar in 100 μ1 of an ice-cooled extraction buffer (0.1 M Tris-HC1, pH 7.5, 1 mM PMSF). Then, 50 μ1 of 3×SDS-PAGE sample buffer (30% glycerol, 3% β-mercaptoethanol, 3% SDS, 0.19 M Tris-HCl, pH 6.8, 0.001% BPB) was added and heated at 100° C. for 5 minutes. The resultant mixture was centrifuged at 12,000 rpm for 5 minutes to recover the supernatant. A portion (15 μ1) of the extracted protein was subjected to SDS-polyacrylamide gel electrophoresis and transferred onto a PVDF membrane (Millipore). Immunoblotting was performed on this membrane using the anti-Er mouse antibody prepared in Example 5 as a primary antibody, anti-mouse immunoglobulin alkaline phosphatase-labeled antibody (Jackson) as a secondary antibody and also an alkaline phosphatase coloring substrate (Wake Pure Chemical Industries) to assay the expression of the ER protein. There were a plurality of plants expressing various amounts of the ER protein. From these plans, those expressing a large quantity of the ER protein were selected and subjected to a hypersensitive reaction test and a fungus resistance test.

### EXAMPLE 7

Hypersensitive Reaction Test of the Tobacco Transformant

Induction of hypersensitive reaction by a soybean elicitor was examined using leaves of the tobacco transformants in which high expression of the ER protein had been confirmed in Example 6, as well as leaves of non-transformed tobacco plants and those tobacco plants transformed with the vector (pBl121) alone as controls.

Leaves of tobacco plants grown in a green house were cut off at the petiole and placed in a light-transmissive plastic box. A piece of silicone tube 5 mm in diameter and 5 mm in height was put on the upper surface of each leaf so that a solution could be retained. This tube piece was allowed to retain a chemically synthesized elicitor (β-D-glucohexaoside) [N. Hong and T. Ogawa (1 990), Tetrahedron Lett. 31:3179; released from Prof. Ogawa of the Institute of Physical and Chemical Research and the University of Tokyo] dissolved in a buffer solution (3 mM sodium bicarbonate, 4 mM sodium acetate, pH 8.0) or the buffer solution alone such that the solution was kept in contact with the surface of each leaf.

The leaves were cultured under excessive moisture to prevent drying of the solution through cycles of 16 hours under light and 8 hours in the dark at 25° C. for 7 days. After the culture, the silicone tube was removed, and the induction of synthesis of tobacco phytoalexin was examined on a UV illuminator (Funakoshi). Nothing could be found from the combination of non-transformed tobacco and the chemically synthesized glucan elicitor or from the combination of tobacco plants transformed with the vector alone and the glucan elicitor. This means that tobacco cannot recognize this glucan elicitor. On the other hand, from the combination of tobacco TF-11-1-15 transformed with the ER gene and the chemically synthesized glucan elicitor, accumulation of a remarkable amount of a fluorescent substance (phytoalexin) was recognized. Also, no changes were observed in controls, i.e., those combinations of the tobacco plants and the buffer solution alone or dionized water.

From these results, it was proved that, if the ER gene can be expressed in a host plant other than soybean, there is a possibility that the transformed host plant may acquire the ability to recognize a glucan elicitor which the non-transformed host cannot recognize. The present invention provides not only a possibility to change the plant recognition of a substance, but also a mechanism by which a plant can recognize plant pathogens such as fungi having a glucan structure in their cell walls or the like. As a result, resistant reactions such as induction of phytoalexin closely involved in disease resistance is elicited in the plant. Elicitation of such resistant reactions is important for breeding disease resistant plants.

### EXAMPLE 8

Fungus Resistance Test of the Tobacco Transformant

The tobacco transformants in which high expression of the ER protein had been confirmed in Example 6 were selfed or crossed with glucanase-expressing tobacco plants (Japanese Unexamined Patent Publication No. 4-320631) to harvest seeds, the subsequent generation.

1) Resistance to Phytophthora nicotianae

A strain conserved at Hokkaido University (subcultured in PDA medium from Difco) was transferred to an oatmeal agar and cultured at 25° C. for 4 days. The growing end portion of the mycelium spreading all over the medium was punched with a cork borer to form mycelium disks, which were used as an inoculant. The oatmeal agar medium used in this experiment was prepared as follows. One hundred grams of oatmeal powder was suspended in 1 liter of water, heated at 58° C. for 1 hour and filtered with gauze. To the filtrate, 20 g of agar was added and autoclave-sterilized. Then, the resultant mixture was dispensed into culture dishes for use as a medium.

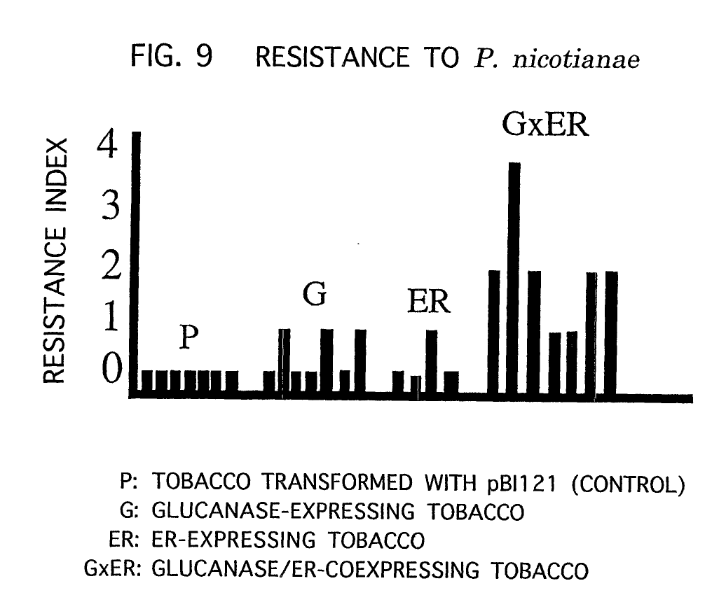

Seedlings obtained from the above-mentioned seeds were tested for expression of ER based on the method described in Example 6. Then, the fungus was inoculated into wounds of those seedlings in which a remarkable expression of ER had been confirmed. Briefly, leaves cut off from tobacco plants about 2 months after germination were placed on a moisturized filter paper in a plastic box. Ten needles tied up together were applied 30 times at one point on both the right and the left side in each of the leaves, yielding punctured wounds in the form of a concentric circle. A small amount of deionized water was applied to the wounds, and then the mycelium disk was inoculated into each wound. Thereafter, the leaves were left at 25° C. for 96 hours. The results of this test are shown in FIG. 9 and Table 3. The resistance of each tobacco leaf tested is shown with a resistance index. The resistance indexes are as follows: “4”: no disease symptom; “3”: up to 25% of the surface bears disease symptoms on both sides of the leaf; “2”: up to 50% of the surface bears disease symptoms on both sides of the leaf; “1”: up to 75% of the surface bears disease symptoms on both sides of the leaf; “0”: 75% or more of the surface bears disease symptoms on both sides of the leaf.

TABLE 3
Resistance to P. nicotianae

| | | | | | | | | | | | | | | |
|---|---|---|---|---|---|---|---|---|---|---|---|---|---|---|
| Tobacco plant | P-1 | 2 | 3 | 4 | 5 | 6 | 7 | G-1 | 4 | 6 | 7 | 8 | 9 | 10 |
| individual No. | | | | | | | | | | | | | | |
| Resistance | 0 | 0 | 0 | 0 | 0 | 0 | 0 | 0 | 1 | 0 | 0 | 1 | 0 | 1 |
| index | | | | | | | | | | | | | | |

| | | | | | | | | | | | |
|---|---|---|---|---|---|---|---|---|---|---|---|
| Tobacco plant | ER-51 | 55 | 56 | 57 | GxER-10 | 11 | 16 | 24 | 28 | 34 | 38 |
| individual No. | | | | | | | | | | | |
| Resistance | 0 | 0 | 1 | 0 | 2 | 4 | 2 | 1 | 1 | 2 | 2 |
| index | | | | | | | | | | | |

P: tobacco transformed with pBI121 (control)
G: glucanase-expressing tobacco
ER: ER-expressing tobacco
GxER: glucanase/ER-coexpressing tobacco

As a result, the formation and expansion of lesions were observed in control pants (plants transformed with pBl121 and plants expressing glucanase alone) whereas the expansion of lesions was not so remarkable in most of the transformants coexpressing glucanase and ER. Therefore, it is believed that the resistance to fungi was improved by the transfer of the ER gene.

2) Resistance to Rhizoctonia solani

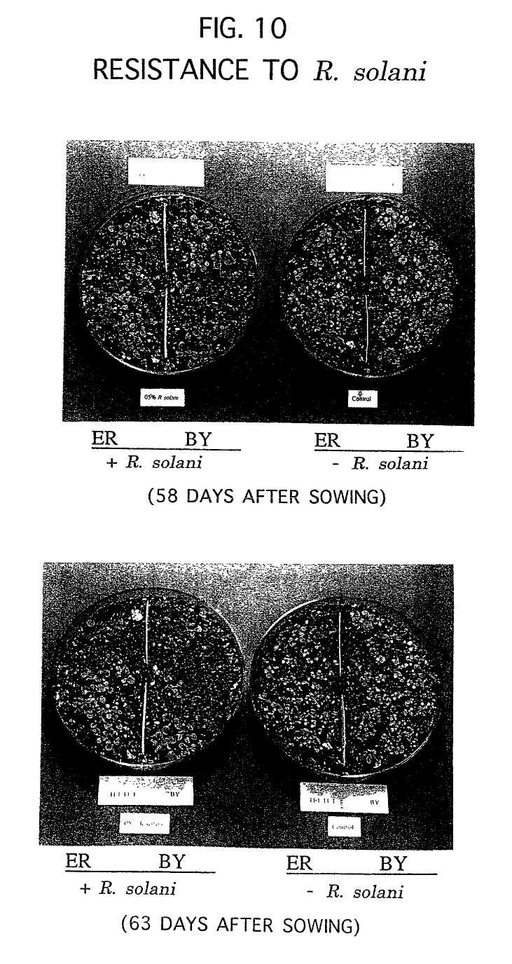

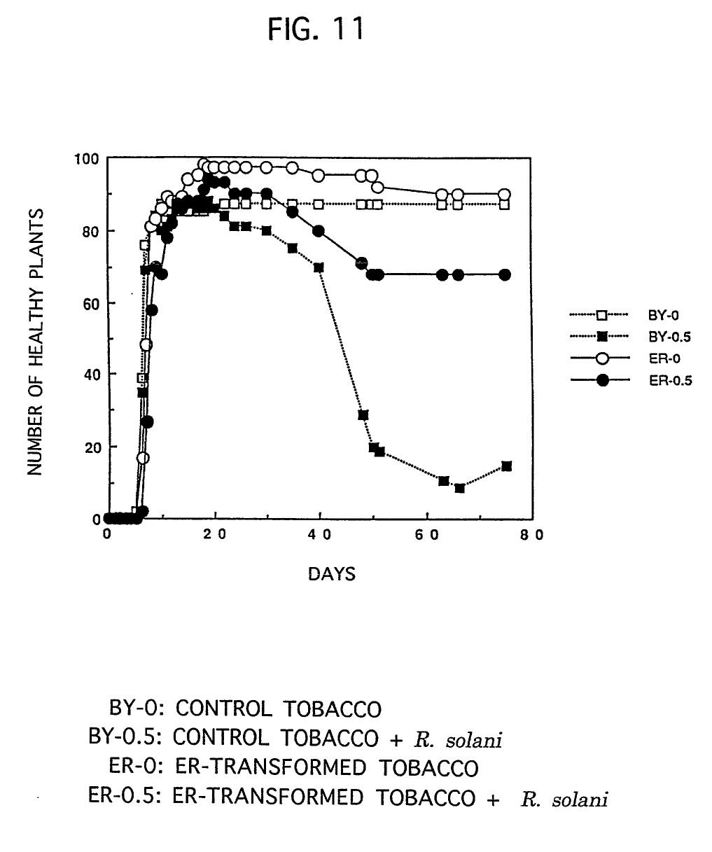

A strain conserved at Gifu University (Rhizoctonia solani AG3 M strain; released from Prof. Hyakumachi of Gifu University; subcultured in PDA medium from Difco) was inoculated into an autoclave-sterilized mixture of barley grains and deionized water (50:50 by volume), cultured at 24° C. for 10 days and dried for 10 days. Then, the barley grains were milled by a coffee maker and mixed well with a soil (river said: vermiculite: peat moss=2:2:1) at a ration of 0.5% (w/w). The rest seeds were sown on this mixture and grown through cycles of 1 6 hours under light and 8 hour in the dark at 25° C. under a humidity of 60-80%. Their growth was observed and, finally, the number of healthy individuals for each of the tested tobacco plants was counted (FIGS. 10 and 11).

As a result, the formation and expansion of lesions were observed in control plants (non-transformed tobacco); the number of healthy individuals decreased sharply; and most of the individuals withered. On the other hand, in ER-expressing transformants, lesions were hardly observed or delay in the developing of disease symptoms was observed (FIG. 10). Therefore, it is believed that the resistance to fungi was improved by the transfer of the ER gene.

3) Fungus Resistance Test using Zoospores of Phytophthora nicotianae

In addition to the fungus resistance test by needle inoculation described in section 1) of Example 8, another fungus resistance test was conducted by inoculating a zoospore suspension. A fungus strain conserved at Hokkaido University (subcultured in PDA medium from Difco) was transferred to an oatmeal agar and cultured at 25° C. in the dark for 1 week. The oatmeal agar was prepared by suspending 100 g of oatmeal powder in 1 liter of water, heating at 58° C. for 1 hour and filtering with gauze; to the filtrate, 20 g of agar was added, autoclave-sterilized and dispensed into culture dishes for use as a medium. From the resultant mycelial flora, disks were punched with a cork borer 6 mm in diameter. The disks were place on 9 cm plastic culture dishes at regular spacings (7 disks/dish). To each dish, 25 ml of a soybean decoction medium (obtained by grinding 400 g of green soybean, filtering the resultant material with gauze and adding distilled water to the filtrate to make a 1 liter solution, followed by autoclave sterilization) was added, and the disks were cultured at 25° C. in the dark for 3 days. After confirming that mycelial mat was formed on almost all over the culture dishes, the medium was discarded. The mycelial mat was washed with an aqueous Petri solution (1 mM KC1, 2 mM Ca(NO₃)_{2,}1.2 mM MgSO_{4,}1 mM KH₂PO₄) three or four times to remove the medium components as completely as possible. Finally, the mycelial mat was washed once with a soil extract (prepared by adding water to 11.5 of field soil to make a volume of 1 liter, filtering the mixture and sterilizing in an autoclave). After swishing water off, the mycelial mat was left at 15° C. under lighting for several days until its surface became slightly dry. It has been found for the first time that this drying treatment is very important for the formation of a large quantity of zoosporangia of the fungus. To the thus formed zoosporangia, the soil extract was added and left to stand at 15° C. under lighting for 2-3 hours. After confirming that a sufficient amount of zoospores was formed, the zoospores were collected to give an inoculant.

The zoospores were inoculated to those plants in which a remarkable expression of ER had been conformed based on the method described in

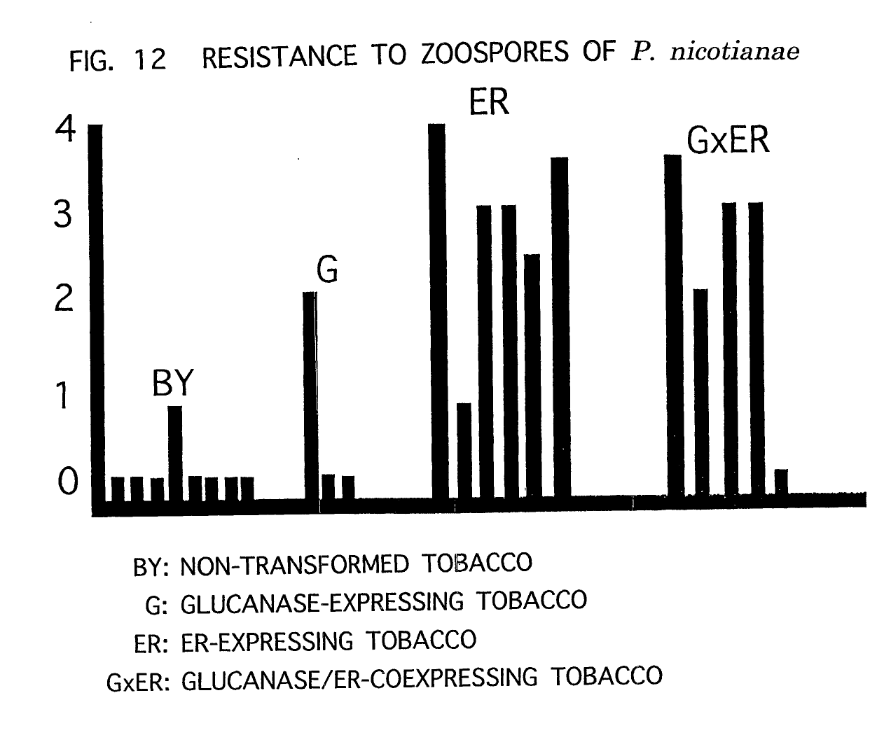

Example 6. Briefly, leaves of tobacco plants about 4 months after germination were cut off and placed on a moisturized filter paper in a plastic box. A silicone ring cut into about 5 mm in length was placed on both the right and the left side of each of the leaves. Then, 100 μ1 of a zoospore suspension (3-5 ×10⁵ zoospores/ml) was added into the silicone ring with a micropipette to thereby inoculate the zoospores to the surface of the tobacco leaf. Then, each leaf was left at 25° C. for 144 hours. The results of this test are shown in FIG. 12 and Table 4.

The resistance of each tobacco leaf tested is shown with a resistance index. The resistance indexes are as follows: “4”: no disease symptom; “3.5”: lesions are restricted to the inoculation site; “3”: up to 25% of the half leaf bears disease symptoms; “2.5”: up to 37.5% of the half leaf bears disease symptoms; “2”: up to 50% of the half leaf bears disease symptoms; “1 ”: up to 75% of the half leaf bears disease symptoms; “0”: more than 75% of the half leaf bears disease symptoms.

TABLE 4
Resistance to Zoospores of P. nicotianae

| Tobacco plant | BY-1 | 2 | 3 | 4 | 5 | 6 | 7 | 8 | G-1 | 2 | 3 |
|---|---|---|---|---|---|---|---|---|---|---|---|
| individual No. | | | | | | | | | | | |
| Resistance | 0 | 0 | 0 | 1 | 0 | 0 | 0 | 0 | 2 | 0 | 0 |
| index | | | | | | | | | | | |
| Tobacco plant | ER-1 | 2 | 3 | 4 | 5 | 6 | GxER-1 | 2 | 3 | 4 | 5 |
| individual No. | | | | | | | | | | | |
| Resistance | 4 | 1 | 3 | 3 | 2.5 | 3.5 | 3.5 | 2 | 3 | 3 | 0 |
| index | | | | | | | | | | | |

BY: non-transformed tobacco (control)
G: glucanase-expressing tobacco
ER: ER-expressing tobacco
GxER: glucanase/ER-coexpressing tobacco

As a result, the formation and expansion of lesions were observed in control plants (non-transformed tobacco and tobacco expressing glucanase alone) whereas the expansion of lesions were inhibited in most of the transformants expressing ER alone or ER and glucanase. Therefore, it is believed that the resistance to fungi was improved by the transfer of the ER gene.

### EXAMPLE 9

Cloning of Novel Kidney Bean Glucanase

1) Kidney bean (Hirasaya Fancy Saitou) seeds (Takayama Seed Co.) were cultured on vermiculite for 12 days and then treated with ethylene for 48 hours according to the method of U. Vogeli et al. [Planta (1 988) 1 74: 364] in order to induce the expression of glucanase. The plants were frozen in liquid nitrogen and stored at −80° C until use. According to the method of Ishida et al. (“Cell Technology Laboratory Manipulation Manual” authored by Ishida and Misawa, Kodansha Scientific), 2.35 mg of total RNA was obtained from 12 g of frozen kidney bean powder.

Subsequently, 1.0 mg of the thus obtained total RNA was used for purification with Oligo (dT) Cellulose (Pharmacia) according to the manual and then 31.5 μg of purified poly(A) +RNA was obtained.

2) Preparation of Kidney Bean cDNA Library

cDNA was synthesized from 5 μg of the poly(A)+RNA with Time Saver cDNA Synthesis Kit (Pharmacia) and random hexamer primers. The synthesized cDNA fragments were ligated to lambda phase vector λ gt10 (Stragene) with T4 DNA ligase (Takara Shuzo Co., Ltd.). Subsequently, the phage vectors were packaged to form phase particles with Gigapack (Stratagene) using a DNA mixed solution to thereby prepare a kidney bean cDNA library of about 1×10⁵ pfu.

3) Preparation of a Screening Probe

Based on the report of B. V. Edington et al. [Plant Molecular Biology (1991) 16:81 ] on the cloning of kidney bean glucanase cDNA, PCR primers were prepared as follows:

sense primer: 5′-CAAATGTTGTGGTAGAGGGATGGCC-3′(SEQ ID NO: 31);

antisense primer: 5′-AAATGTTTCTCTATCTCAGGACTC-3′(SEQ ID NO: 32).

An RT-PCR was performed with these primers according to the method of Ishida (“Gene High Expression Experiment Manual”, Ishida and Ando (Eds.), Kodansha Scientific) to give a PCR fragment of about 300 bp. This fragment was subcloned into the EcoRV site of pBluescript SKll+(Stratagene). For the cDNA synthesis, 1 mg of total RNA and 0.5 mg of random hexamer primers (Takara Shuzo Co., Ltd.) were used. The DNA sequence of the insert (0.3 kbp) in the subclone plasmid was determined. As a result, the DNA sequence was found to be identical with the glucanase cDNA reported by B. V. Edington et al. (supra). This plasmid DNA was digested with Hindlll and EcoRV, and fractionated by agarose gel electrophoresis. The insert DNA was purified with Gene Clean II (Bio 101) to give a probe for screening the kidney bean cDNA library prepared in 2) above.

4) Screening and Cloning of the Library by Hybridization

The DNA fragment obtained as a screening probe was labelled with [α- ³²P]dCTP using Megaprime DNA labelling kit (Amersham) according to the manual, and the reaction solution was subjected to the subsequent hybridization experiment.

E. coli C600 hfl (Invitrogen) was transfected with the kidney bean cDNA library prepared in 2) above, and inoculated into L medium supplemented with 10 mg/ml MgCl2 in a culture dish about 15 cm in diameter to form a total of about 1×10⁵ plaques. The plaques were blotted to a nylon membrane (GeneScreen (+); NEN DuPont). The membrane was reacted with the ³²P-dCTP-labelled glucanase cDNA fragment, and positive phages detected by autoradiography were screened again in the same manner to give one phase clone.

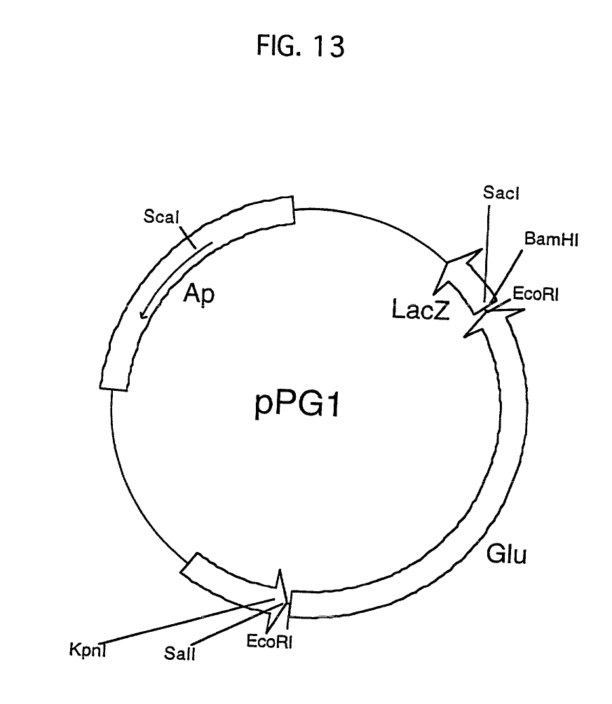

The λ phage DNA was purified with Lambda Sorb (Promega) from a solution of the positive clone isolated in the hybridization experiment. Five micrograms of this DNA was digested with EcoRl and fractionated by 1% agarose gel electrophoresis to cut out an about 1.2 kb band. This band was treated with Gene Clean II (Bio 101) to recover a solution containing the DNA, which was subcloned into the EcoRl site of vector pBluescriptll KS+(Stratagene). FIG. 13 shows the structure of plasmid pPG1.

5) Determination of the Nucleotide Sequence of the DNA Coding for Kidney Bean Glucanase

The DNA of the plasmid into which the glucanase cDNA had been cloned was sequenced in both orientations with a fluorescence sequencer by preparing a series of plasmids having deletions at intervals of ca. 200-300 bp, using Kilosequence Deletion Kit (Takara Shuzo Co., Ltd.). The resultant nucleotide sequence for the DNA is shown in SEQ ID NO: 33. As a result, the DNA fragment was found to contain an ORF of 993 bp starting from a nucleotide sequence corresponding to an amino acid sequence that was predictably to be a signal sequence; it is presumed that 331 amino acid residues are encoded in the ORF. This amino acid sequence is shown in SEQ ID NO: 34.

As a result of a search using BLAST Protein Search, the amino acid sequence deduced from the resultant nucleotide sequence was found to be a completely novel sequence. Compared with the amino acid sequence reported previously by B. V. Edington et al. [Plant Molecular Biology (1991) 16:81], there was 49% homology (excluding the portion which appeared to be a signal sequence). Besides, it had 51% homology in full length to the soybean-derived amino acid sequence reported by Y. Takeuchi et al. [Plant Physiol. (1 990) 93:673]. Since the deduced amino acid sequence exhibits high homology to the amino acid sequences of the previously reported glucanases, it was expected that the resultant DNA sequence would also encode a glucanase. It was also expected that, unlike previously reported kidney bean glucanases, the glucanase under discussion was of an extracellular secreting type since the deduced amino acid sequence had a would-be signal sequence at its N-terminal while lacking a would-be vacuole targeting sequence at its C-terminal.

### EXAMPLE 10

Expression of the Kidney Bean Glucanase

1) Construction of Plasmid pGST-PG1

PCR sense primer: 5′-GGAATTCCGAATCTGTGGGTGTGTGT TAT-3′(SEQ ID NO: 35) and antisense primer: M13 reverse sequence primer (SEQ ID NO: 36) were designed so that the kidney bean glucanase sequence [excluding the signal sequence, i.e. Met(1)-Val(21) at the N-terminal] could be ligated downstream from glutathione-S-transferase expression vector (Pharmacia; pGEX-4T-3) in an in-frame fashion. With these primers, a PCR was performed on 0.1 mg of a template plasmid pPG1 DNA using Ex-Taq DNA polymerase (Takara Shuzo) (annealing temperature =50° C.; 20 cycles). The amplified PCR fragments were digested with EcoRI and fractionated by agarose gel electrophoresis to give a DNA fragment of about 1 kbp. This fragment was purified with Gene Clean II and subeloned into the EcoRi site of pGEX-4T-3 expression vector using JM109 competent cells (Toyobo) (pGST-PG1).

Expression in E. Coli BL21

The plasmid DNA was purified from the subclone obtained in 1) above and re-transferred into E. coli BL21 competent cells (Molecular Cloning, Cold Spring Harbor Laboratory Press). The E. coli BL21 was released from Prof. Masayuki Yamamoto, the Department of Science, University of Tokyo.

3) Purification of GST-Fused Glucanase

An overnight culture (4 ml) of the E. coli obtained in 1) above was transferred into 200 ml of 2 ×YT medium containing 100 mg/ml ampicillin and cultured at 37° C. for 1.5 hours. IPTG (Takara Shuzo Co., Ltd.) was added thereto to give a final concentration of 0.1 mM, and the cells were cultured for another 4 hours. The cells were harvested from the culture solution by centrifugation at 10,000 rpm and 4° C. for 10 minutes. According to the Gene Expression Experiment Manual (supra), approximately 1 mg of glutathione-S-transferase/glucanase fusion protein (molecular weight: about 62 kDa) was purified using Glutathione Sepharose (Pharmacia).

4) Determination of Glucanase Activity

The glucanase activity of the purified glutathione-S-transferase/glucanase fusion protein was determined by the following procedure. Briefly, an enzymic reaction solution was incubated at 37° C. The reaction was terminated at 0, 10, 20 and 30 minutes from the start of the reaction, and the liberated glucose was quantitatively determined by the method of Nelson [N. Nelson, J. Biol. Chem. (1944) 153, 375]. The enzymatic reaction solution was composed of 0.5 ml of 50 mM acetate buffer (pH 5.5), 2.5 mg of laminarin as a substrate, and 0, 0.51 or 5.1 μg of the glutathione-S-transferase/glucanase fusion protein as an enzyme.

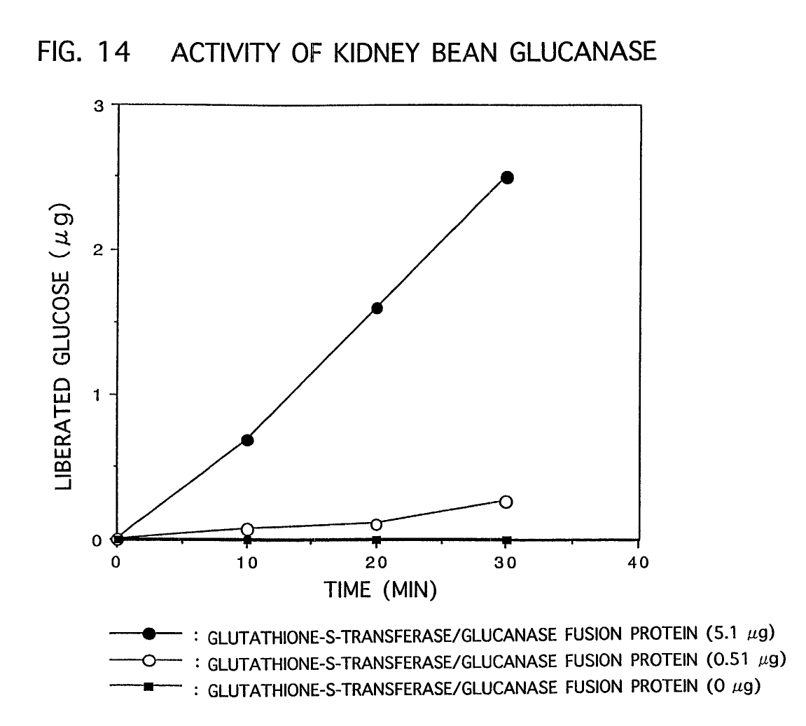

As a result, glucose was liberated from laminarian in a manner dependent on both enzyme concentration and reaction time (see FIG. 14). Thus, it was made clear that the newly cloned cDNA has glucanase activity. This suggests the possibility that the glucanase under consideration can also be utilized for improving plants' resistance to fungi, like the soybean-derived glucanase encoded by SEQ ID NO: 4.

### EXAMPLE 11

Detection of Elicitor Receptor (ER) Homologous Genes in Other Plants

To identify the presence of homologous genes having structures similar to the elicitor receptor (ER) gene isolated from a soybean variety, Green Homer, described in Example 2, genomic DNAs of the following two groups were subjected to Southern hybridization:

1) Soybean varieties: Acme, Flambeau, Harosoy, Harosoy 63, and Merit; and

2) Non-soybean plant species: bean, mung bean, pea, potato, Arabidopsis, tobacco, tomato, rice, maize, and Phytophthra megasperma f. sp. glycinea (which is a bacterium containing an elicitor).

As positive controls, Green Homer and soybean (Green Homer) were used in groups 1 and 2, respectively.

As general, molecular-biological procedures, the methods of Sambrook et al. (Molecular Cloning, Cold Spring Harbor Laboratory Press, New York, 1 989) were employed. Extraction of DNAs from the plants was conducted using Dneasy (Qiagen). Part of each DNA was cleaved with the restriction enzyme EcoRI, and fractionated by electrophoresis on 1% agarose gel. The hybridization was conducted using ³²P-labeled cDNA derived from Green Homer as a probe, according to the protocol of Hybond N+(Amersham). Wash conditions were as follows:

2xSSC/0.1% SDS, room temp., 10 min. (twice);

2XSSPE/0.1% SDS, 42° C., 45 min (once).

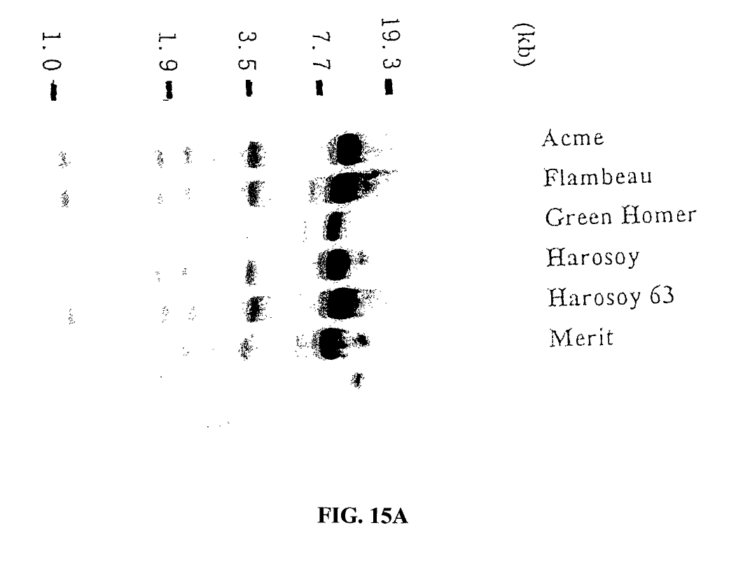

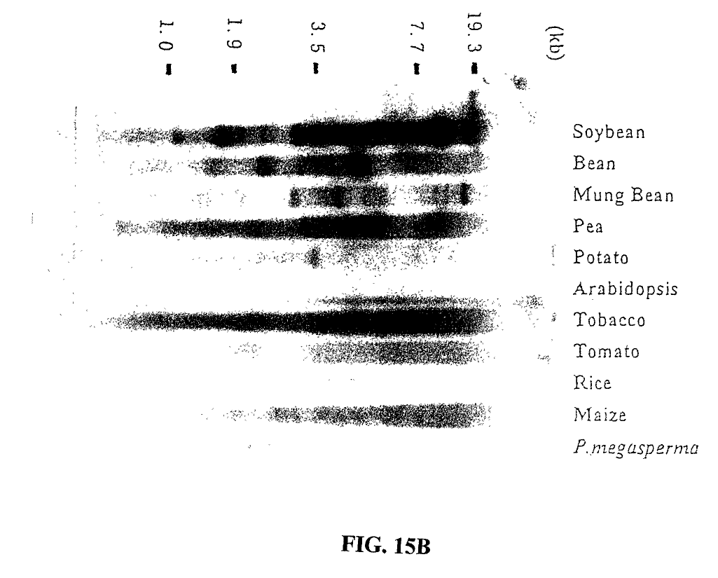

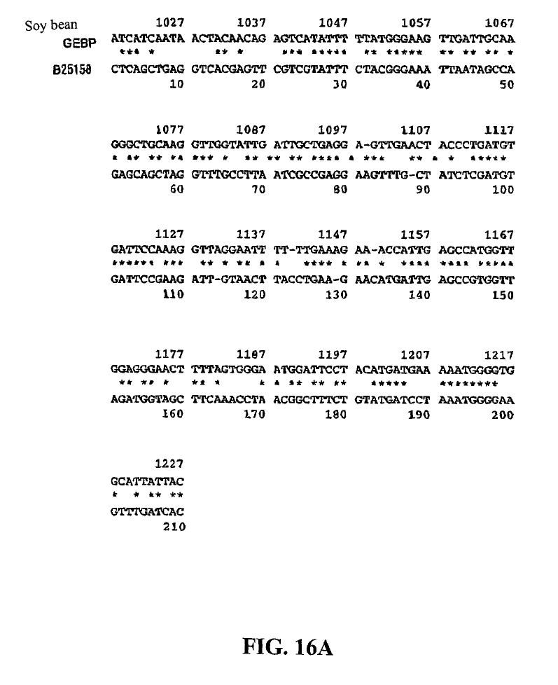

The results of the hybridization indicated that all soybean varieties, i.e., Acme, Flambeau, Green Homer (positive control), Harosoy, Harosoy 63 and Merit (FIG. 15(A)), and bean and mung bean (FIG. 15(B)), have clear homologous genes. Hybridization signal was similarly observed in pea, potato, tobacco, tomato, and maize, etc., indicating that there exist structurally similar genes in these plants.

### EXAMPLE 12

Detection of glucan elicitor binding protein (GEBP) gene in Arabidopsis

To examine whether a gene homologous to soybean glucan elicitor binding protein (GEBP) gene is present in other plants, we have studied Arabidopsis as an experimental material.

Materials and Methods

1) Sample Plant

Arabidopsis (Columbia) seeds were sowed over pots containing culture soil, over which vermiculite had been layered. The seeds were supplied with 1000 x Hyponex solution every week and cultured under light at 20° C. On day 20, all the plants were harvested and used to extract the genomic DNA or RNA.

2) Plaque Hybridization

E. coli strain XL-1 blue MRF was infected with Uni-ZAP XR phage containing the cDNA library (Stratagene) of Arabidopsis to form plaques. The resulting approximately 5×10⁴ plaques were blotted onto a nylon membrane (Hybond-N+; Amersham) followed by hybridization with a random primer labelling kit (Takara Shuzo Co., Ltd.) using 32P-labeled cDNA for soybean GEBP as a probe. Hybridization was performed at 37° C. using a solution containing 20% formamide, 5×Denhardt's reagent, 5 ×SSPE, 0.1% SDS, and 100 μg/ml denatured salmon sperm DNA. The membrane was washed twice with 3 ×SSC and 0.1% SDS for 30 minutes at 37° C., followed by washing with 1 ×SSC and 0.1% SDS for 30 minutes at 37° C. Then the membrane was exposed to X-ray film.

3) Search of GEBP sequences in Arabidopsis

Arabidopsis DNA sequences having partial homology with that encoding soybean GEBP were searched using a database. As a result, two terminal sequences of BAC clone, B25158 and B24124, were shown to have homology with soybean GEBP.

4) PCR

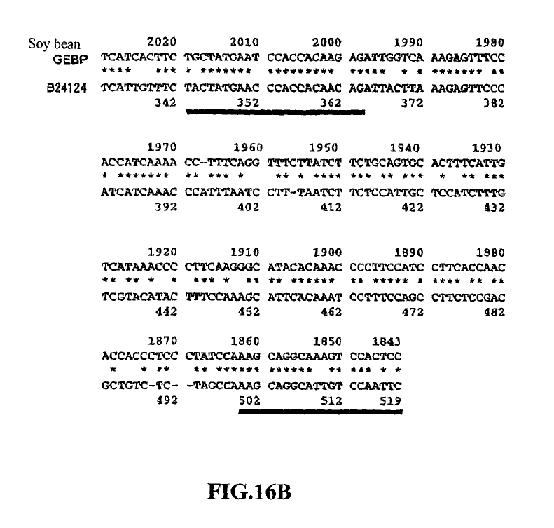

Template DNA was isolated from Arabidopsis by the rapid isolation method (PCR Experimental Protocols for Plants, Shu-jun-sha publishing, Tokyo, Japan). Two primers B25158a and B25158b were designed for B25158, and one primer was designed for B24124 (FIG. 16(A), (B)). Primer structures are as follows:

B25158a primers>

Forward primer: 5′GAGGTCAGGAGTTCGTCGTA-3′(SEQ ID NO: 37)

Reverse primer: 5′-AGCTACCATCTAACCACGGC-3″(SEQ ID NO. 38)

<B25158b primers>

Forward primer: 5′-TCAGCTGAGGTCACGAGTTC-3′(SEQ ID NO: 39)

Reverse primer: 5TGATCAAACTTCCCCATTTAGG-3′(SEQ ID NO. 40)

<B24124 primers>

Forward primer: 5′-TACTATGAACCCACCACAACAG-3′(SEQ ID NO:41)

Reverse primer: 5′GAATTGGACAATGCCTGCCTGCTT-E′ (SEQ ID NO:42)

Reaction solution 50 μ was prepared to contain 100 ng of Arabidopsis DNA, 5 μl of 2.0 mM dNTPs, 5 μl of 10 ×PCR buffer, 0.5 μl each of forward and reverse primers (concentration: 50 pM), 0.5 μl (2.5 units) of Taq DNA polymerase (Ampli Tag Gold, Perkin-Elmer). Following thermal denaturation of the template for 9 min at 95° C. for 30 sec, 52° C. for 1 min, and 72° C. for 1 min. Additional incubation was then performed at 720 min. At the end of the reaction, the reaction mixture was kept at 4° C. PCR was performed using a Gene Amp PCR system 2400 (perkin-Elmenter). Following reaction, 10 μ or products was resolved by gel electrophoresis on 4.5A agarose (Nusieve GTG, agarae, FMC Fmc BioProducts). The remaining reaction mixture was subjected to ethanol precipitation to recover the products.

5) RT-PCR

Total RNA was isolated from Arabidopsis plant by a RNA simple isolation method called the GTC method (PCR Experimental Protocols for Plants, Shu-jun-sha publishing, Tokyo, Japan). Then mRNA was isolated from the total RNA using an Oligotex-dt30 <Super>(JSR). This mRNA was used as a template RNA. Reaction was performed using RT-PCR Beads (Amersham Pharmacia Biotech). Reaction solution 50 μl was prepared to contain 10 ng of Arabidopsis RNA, 0.25 μl (concentration: 25 pM) forward primer as a first strand primer, and 0.25 μl (concentration: 25 pM) each of forward and reverse primers. The above three types of primers were used. Reverse transcription from RNA to DNA was performed at 42° C. for 30 minutes, and thermal denaturation of the template at 95° C for 5 minutes. Then B25158a primers were used to perform PCR under the conditions: 32 cycles of 95° C. for 30 sec, 52° C. for 1 min, and 72° C for 1 min, and PCR using B2515b and B24124 primers was performed under the conditions: 32 cycles of 95° C. for 30 sec, 48° C. for 1 min, and 72° C. for 1 min. Additional incubation was performed at 72° C. for 3 min. At the end of the reaction, the reaction mixture was kept at 4°. Subsequently, the products were detected and recovered as described above.

6) Determination and Comparison of Nucleotide Sequences

DNAs were recovered from bands detected by PCR and T-PCR, and then inserted into pCRII vectors (Invitrogen). After introduction of each plasmid vector into E. coli, plasmids were extracted from the resulting E. coli clones using a Wizard Plus Minipreps DNA Purification System (Promega). Then PCR was performed using M13 primer and Dye Terminator Ready Reaction Mix (Applied Biosystems). Next the nucleotide sequence within the insertion region was determined using an ABl373A DNA sequencer (Applied Biosystems).

The nucleotide sequences and amino acid sequences of the determined band sequence, B25158, and soybean GEBP were compared and analyzed using GENETYX.

Results

1) Plaque hybridization

To isolate an Arabidopsis gene having homology with soybean GEBP, the Arabidopsis cDNA library was subjected to a screening using soybean cDNA as a probe. However, because the obtained clones did not show any positive signal, the sequence of an Arabidopsis gene having homology with soybean GEBP was determined by search of a database.

2) Search of the Putative Sequence of Arabidopsis GEBP

From the database search, it was found that there exists a BAC clone terminal sequence having high homology with soybean GEBP. B25158 had homology with the central portion of soybean GEBP having an elicitor-binding region. The nucleotide sequence of B25158 consisted of 207 bp and had homology of 68% with that of soybean GEBP, and the deduced amino acid sequence of B25158 had homology of 64% with that of soybean GEBP (FIG. 16(A)). On the other hand, B241 24 had homology with a C-terminal portion of soybean GEBP. The nucleotide sequence of B24124 consisted of 515 bp, and the nucleotide sequence of positions 333 to 515 had homology of 73% with that of soybean GEBP, and the deduced amino acid sequence of B24124 had homology of 63% with that of soybean GEBP (FIG. 16 (B)). The two sequences were both derived from genomic DNA, and it is unknown whether they are actually transcribed or not. Hence, RT-PCR was performed using Arabidopsis RNA to confirm that these sequences were transcribed.

3) PCR

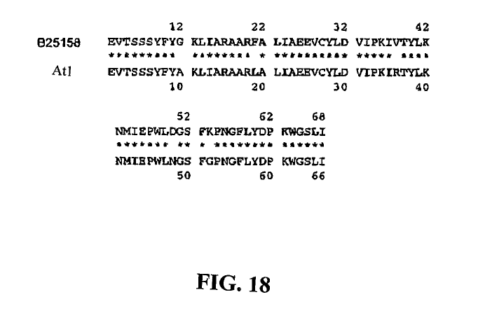

Before performing RT-PCR, primers for B25158 or B24124 were designed, and PCR was performed using Arabidopsis DNA as a template, to confirm whether the sequence of interest can be amplified with these primers. It was predicted that a band of 156 bp, 199 bp or 172 bp would be detected using B25158a primers, B25158b primers or B24124 primers, respectively. As a result of PCR using the 3 types of primers, bands having the predicted size were detected (FIG. 18). Then, we cloned the three bands in order to determine their nucleotide sequences, and found that all of the nucleotide sequences matched to the sequences to be amplified.

4) RT-PCR, and Determination and Comparison of Nucleotide Sequences

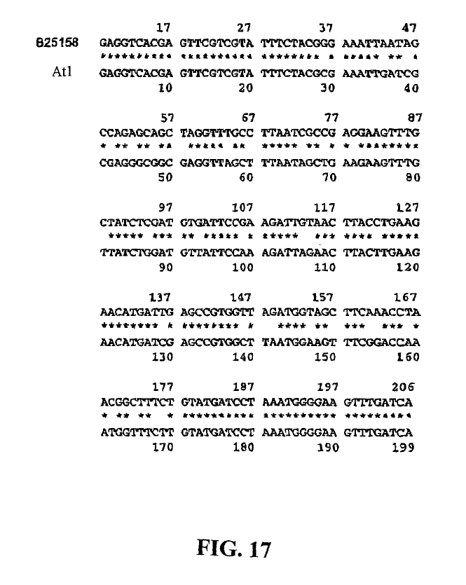

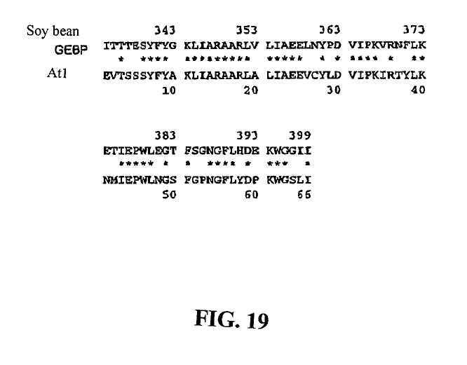

RT-PCR was performed using the above 3 types of primers and Arabidopsis mRNA as a template. When B25158a primers were used, two bands were detected at different positions above and below 200 bp, the bands being both larger than a predicted size, 156 bp. Through analysis of the nucleotide sequences of both bands, it was found that Arabidopsis DNA had low homology with soybean GEBP in respect of both the nucleotide sequence and the amino acid sequence (approx. 20% homology). When B24124 primers were used, no bands were detected. When B25158b primers were used, one thinner band (Atl) was detected at the expected position of approximately 200 bp. As a result of analysis of its nucleotide sequence, although the sequence of Atl did not perfectly match to that of B25158, it had high homology with B25158 and soybean (GEBP) in respect of both the nucleotide sequence and the amino acid sequence. The nucleotide sequence of Atl had 84% homology with that of B25158 (FIG. 17), and the deduced amino acid sequence of Atl had 92% homology with that of B25158 (FIG. 18). Furthermore, the nucleotide sequence of Atl had 64% homology with that of soybean GEBP, and the deduced amino acid sequence of Atl had 67% homology with that of soybean GEBP (FIG. 19).

It is assumed that because there exists a gene having high homology with soybean GEBP in Arabidopsis, the gene is expressed in the plant. It is known that Atl has homology with the central portion of soybean GEBP, to which glucan is considered to bind. The above-mentioned experiments indicate that there is a possibility that DNA in a portion having homology with a glucan-binding portion is expressed in Arabidopsis and functions as GEBP.

Industrial Applicability

According to the present invention, a plant into which DNA sequence coding for a glucan elicitor receptor has been transferred and which expresses the glucan elicitor receptor, as well as a method for producing the plant, is provided. The plants of the present invention have high resistance to fungi.

What is claimed is:

1. A method for producing a plant with enhanced resistance to a pathogenic fungus, comprising transforming a plant with an expression vector comprising a DNA encoding a glucan elicitor receptor operabliy linked to a heterologous promoter, selecting a plant which has said DNA, and recovering the selected plant.

2. The method of claim 1 wherein said DNA is derived from a plant.

3. The method of claim 1 wherein said DNA is derived from a dicotyledonous plant or a monocotyledonous plant.

4. THe method of claim 1 wherein said DNA codes for a glucan elicitor receptor comprising (i) an amino acid sequence as shown in SEQ ID NO:1, or (ii) an amino acid sequence comprising residues 239-442 of SEQ ID NO:1, or (iii) a fragment thereof which exhibits glucan elicitor receptor activity.

5. The method of claim 1 wherein said method further comprises incorporating a DNA encoding a glucanase into a chromosome of said plant, and expressing said DNA in said plant, and recovering the resulting plant.

6. The method of claim 5 wherein the DNA is derived from a plant.

7. The method of claim 5 wherein the DNA is derived from a dicotyledonous plant or a monocotyledonous plant.

8. The method of claim 5 wherein the glucanase is of an extracellualry secreted type.

9. The method of claim 5 wherein the DNA encoding glucanase comprises a nucleotide sequence encoding an amino acid sequence as shown in SEQ ID NO:3 or SEQ ID NO:34 or a fragment thereof which exhibits glucanase activity.

10. A method for producing a progeny plant with resistance to a pathogenic fungus, comprising transforming an ancestral plant with an expression vector comprising a DNA encoding a glucan elicitor receptor operably linked to a heterologous promoter selecting a plant which has said DNA incorporated into a chromosome of said plant and which expresses said DNA, and obtaining progeny from the selected plant through cultivation and/or breeding.


SEQUENCE LISTING (52 sequences)

SEQ ID NO: 1 - protein, 667 aa, Glycine max
VNIQTNTSYIFPQTQSTVLPDPSKFFSSNLLSSPLPTNSFFQNFVLKNGDQQEYIHPYLIKSSNSSLSLSYPSRQASSAVIFQVFNPDLTISAPQGPKQGPPGKHLISSYSDLSVTLDFPSSNLSFFLVRGSPYLTVSVTQPTPLSITTIHSILSFSSNDSNTKYTFQFNNGQTWLLYATSPIKLNHTLSEITSNAFSGIIRIALLPDSDSKHEAVLDKYSSCYPVSGKAVFREPFCVEYNWEKKDSGDLLLLAHPLHVQLLRNGDNDVKILEDLKYKSIDGDLVGVVGDSWVLKTDPLFVTWHSIKGIKEESHDEIVSALSKDVESLDSSSITTTESYFYGKLIARAARLVLIAEELNYPDVIPKVRNFLKETIEPWLEGTFSGNGFLHDEKWGGIITQKGSTDAGGDFGFGIYNDHHYHLGYFIYGIAVLTKLDPAWGRKYKPQAYSIVQDFLNLDTKLNSNYTRLRCFDPYVLHSWAGGLTEFTDGRNQESTSEAVSAYYSAALMGLAYGDAPLVALGSTLTALEIEGTKMWWHVKEGGTLYEKEFTQENRVMGVLWSNKRDTGLWFAPAEWKECRLGIQLLPLAPISEAIFSNVDFVKELVEWTLPALDREGGVGEGWKGFVYALEGVYDNESALQKIRNLKGFDGGNSLTNLLWWIHSRSDE

SEQ ID NO: 2 - DNA, 2004 bp, Glycine max
gttaacatccaaaccaatacatcttacatcttccctcaaacacaatccactgttcttcctgatccctccaaattcttctcctcaaaccttctctcaagtccactccccacaaactctttcttccaaaactttgtcctaaaaaatggtgaccaacaagaatacattcatccttacctcatcaaatcctccaactcttccctctctctctcatacccttctcgccaagccagttcagctgtcatattccaagtcttcaatcctgatcttaccatttcagccccacaaggtcccaaacaaggtccccctggtaaacaccttatctcctcctacagtgatctcagtgtcaccttggatttcccttcttccaatctgagcttcttccttgttaggggaagcccctatttgactgtgtctgtgactcaaccaactcctctttcaattaccaccatccattccattctctcattctcttcaaatgactccaacaccaagtacacctttcagttcaacaatggtcaaacatggcttctttatgctacctcccccatcaagttgaaccacaccctttctgagataacttctaatgcattttctggcataatccggatagctttgttgccggattcggattcgaaacacgaggctgttcttgacaagtatagttcttgttaccccgtgtcaggtaaagctgtgttcagagaacctttctgtgtggaatataactgggagaagaaagattcaggggatttgctactcttggctcaccctctccatgttcagcttcttcgtaatggagacaatgatgtcaaaattcttgaagatttaaagtataaaagcattgatggggatcttgttggtgttgtcggggattcatgggttttgaaaacagatcctttgtttgtaacatggcattcaatcaagggaatcaaagaagaatcccatgatgagattgtctcagccctttctaaagatgttgagagcctagattcatcatcaataactacaacagagtcatatttttatgggaagttgattgcaagggctgcaaggttggtattgattgctgaggagttgaactaccctgatgtgattccaaaggttaggaattttttgaaagaaaccattgagccatggttggagggaacttttagtgggaatggattcctacatgatgaaaaatggggtggcattattacccaaaaggggtccactgatgctggtggtgattttggatttggaatttacaatgatcaccactatcatttggggtacttcatttatggaattgcggtgctcactaagcttgatccagcatggggtaggaagtacaagcctcaagcctattcaatagtgcaagacttcttgaacttggacacaaaattaaactccaattacacacgtttgaggtgttttgacccttatgtgcttcactcttgggctggagggttaactgagttcacagatggaaggaatcaagagagcacaagtgaggctgtgagtgcatattattctgctgctttgatgggattagcatatggtgatgcacctcttgttgcacttggatcaacactcacagcattggaaattgaagggactaaaatgtggtggcatgtgaaagagggaggtactttgtatgagaaagagtttacacaagagaatagggtgatgggtgttctatggtctaacaagagggacactggactttggtttgctcctgctgagtggaaagagtgtaggcttggcattcagctcttaccattggctcctatttctgaagccattttctccaatgttgactttgtaaaggagcttgtggagtggactttgcctgctttggatagggagggtggtgttggtgaaggatggaaggggtttgtgtatgcccttgaaggggtttatgacaatgaaagtgcactgcagaagataagaaacctgaaaggttttgatggtggaaactctttgaccaatctcttgtggtggattcatagcagaagtgatgaatag

SEQ ID NO: 3 - protein, 347 aa, Glycine max
MAKYHSSGKSSSMTAIAFLFILLITYTGTTDAQSGVCYGRLGNNLPTPQEVVALYNQANIRRMRIYGPSPEVLEALRGSNIELLLDIPNDNLRNLASSQDNANKWVQDNIKNYANNVRFRYVSVGNEVKPEHSFAQFLVPALENIQRAISNAGLGNQVKVSTAIDTGALAESFPPSKGSFKSDYRGAYLDGVIRFLVNNNAPLMVNVYSYFAYTANPKDISLDYALFRSPSVVVQDGSLGYRNLFDASVDAVYAALEKAGGGSLNIVVSESGWPSSGGTATSLDNARTYNTNLVRNVKQGTPKRPGAPLETYVFAMFDENQKQPEFEKFWGLFSPITKQPKYSINFN

SEQ ID NO: 4 - DNA, 1044 bp, Glycine max
atggctaagtatcattcaagtgggaaaagctcttccatgactgctatagccttcctgtttatccttctaatcacttatacaggcacaacagatgcacaatccggggtatgttatggaagacttggcaacaacttaccaacccctcaagaagttgtggccctctacaatcaagccaacattcgcaggatgcgaatctacggtccaagcccagaagtcctcgaagcactaagaggttccaacattgagcttttgctagacattccaaatgacaacctcagaaacctagcatctagccaagacaatgcaaacaaatgggtgcaagacaacatcaaaaactatgccaacaatgtcagattcagatacgtttcagtgggaaatgaagtgaaacccgaacactcatttgcacaatttctagtgcctgcattggaaaacattcagagggccatttctaatgctggccttggaaaccaagtaaaagtttccactgccattgatactggtgccttggcagaatcattcccaccatcaaagggttccttcaaatctgattatagaggagcatatcttgatggtgtcatcagatttctagtgaacaataatgccccattaatggttaatgtgtactcttacttcgcttacactgcaaaccctaaggacattagtcttgactatgcactttttaggtctccttcggtggtagtgcaagatggttcacttggttaccgtaacctctttgatgcttcggttgatgctgtttatgctgcattggagaaagcaggaggagggtcattgaacatagttgtgtctgagagtggatggccttcttctggtggaactgcaacttcacttgataatgcaagaacttacaacacaaacttggttcggaatgtgaagcaaggaacccctaaaaggcctggtgcaccccttgaaacttatgtgtttgccatgtttgatgaaaatcagaagcagccagagtttgaaaaattttgggggctcttttctcctataactaagcagcccaaatactcgattaatttcaattaa

SEQ ID NO: 5 - protein, 13 aa, Glycine max
VNIQTNTSNISPQ

SEQ ID NO: 6 - protein, 14 aa, Glycine max
KSIDGDLVGVVGDS

SEQ ID NO: 7 - protein, 17 aa, Glycine max
KYKPQAYSIVQDFLNLD

SEQ ID NO: 8 - protein, 13 aa, Glycine max
KTDPLFVTWHSIK

SEQ ID NO: 9 - DNA, 17 bp, Artificial Sequence
  Description of Artificial Sequence Primer
aaragyathgayggnga

SEQ ID NO: 10 - DNA, 13 bp, Artificial Sequence
  Description of Artificial Sequence Primer
wrtcnccnacnac

SEQ ID NO: 11 - DNA, 17 bp, Artificial Sequence
  Description of Artificial Sequence Primer
gtnaayaaratncarac

SEQ ID NO: 12 - DNA, 17 bp, Artificial Sequence
  Description of Artificial Sequence Primer
arrttnagraartcytc

SEQ ID NO: 13 - DNA, 24 bp, Artificial Sequence
  Description of Artificial Sequence Primer
aagtayaagccrcaagcctattca

SEQ ID NO: 14 - DNA, 17 bp, Artificial Sequence
  Description of Artificial Sequence Primer
atcgccracaacmccaa

SEQ ID NO: 15 - DNA, 54 bp, Artificial Sequence
  Description of Artificial Sequence Selection probe
ggaattcgagctcggtacccgggggatcctctagagtcgacctgcaggcatgca

SEQ ID NO: 16 - DNA, 58 bp, Artificial Sequence
  Description of Artificial Sequence Selection probe
ccttaagctcgagccatgggccccctaggagatctcagctggacgtccgtacgttcga

SEQ ID NO: 17 - DNA, 26 bp, Artificial Sequence
  Description of Artificial Sequence Primer
atggatccatggttaacatccaaacc

SEQ ID NO: 18 - DNA, 26 bp, Artificial Sequence
  Description of Artificial Sequence Primer
atggatccgaatataactgggagaag

SEQ ID NO: 19 - DNA, 26 bp, Artificial Sequence
  Description of Artificial Sequence Primer
atggatccccagcatggggtaggaag

SEQ ID NO: 20 - DNA, 28 bp, Artificial Sequence
  Description of Artificial Sequence Primer
tagtcgactacttctcccagttatattc

SEQ ID NO: 21 - DNA, 28 bp, Artificial Sequence
  Description of Artificial Sequence Primer
tagtcgactacttcctaccccatgctgg

SEQ ID NO: 22 - DNA, 28 bp, Artificial Sequence
  Description of Artificial Sequence Primer
tagtcgactattcatcacttctgctatg

SEQ ID NO: 23 - DNA, 26 bp, Artificial Sequence
  Description of Artificial Sequence Primer
atggatccgccccacaaggtcccaaa

SEQ ID NO: 24 - DNA, 26 bp, Artificial Sequence
  Description of Artificial Sequence Primer
atggatccaatgactccaacaccaag

SEQ ID NO: 25 - DNA, 26 bp, Artificial Sequence
  Description of Artificial Sequence Primer
atggatccgaatataactgggagaag

SEQ ID NO: 26 - DNA, 28 bp, Artificial Sequence
  Description of Artificial Sequence Primer
tagtcgactacttcctaccccatgctgg

SEQ ID NO: 27 - DNA, 34 bp, Artificial Sequence
  Description of Artificial Sequence Selection probe
ctagaggatccggtacccccggggtcgacgagct

SEQ ID NO: 28 - DNA, 26 bp, Artificial Sequence
  Description of Artificial Sequence Selection probe
cgtcgaccccgggggtaccggatcct

SEQ ID NO: 29 - DNA, 20 bp, Artificial Sequence
  Description of Artificial Sequence Primer
caccttcagcaacaatggtt

SEQ ID NO: 30 - DNA, 20 bp, Artificial Sequence
  Description of Artificial Sequence Primer
ctattcatcacttctgctat

SEQ ID NO: 31 - DNA, 24 bp, Artificial Sequence
  Description of Artificial Sequence Primer
caaatgttgtggtgagggatggcc

SEQ ID NO: 32 - DNA, 24 bp, Artificial Sequence
  Description of Artificial Sequence Primer
aaatgtttctctatctcaggactc

SEQ ID NO: 33 - DNA, 996 bp, Phaseolus vulgaris
atgtctgccttattgctgcttcttggagtattatcttccactggagtactgcttactggggtagaatctgtgggtgtgtgttatggaggaaatggaaacaatctaccaacaaagcaagcagtggtgaatctctacaaatcaaacggaattggcaaaatccgtttatactatccagatgaaggtgcccttcaagccctcagaggttcaaacatagaagtgatacttgctgttcctaatgatcaacttcaatctgtctccaacaatggaagtgcaacaaattgggtcaacaattacgtgaaaccctatgcaggaaacgtgaaattgaagtacattgcagttggcaacgaagttcaccctggtgatgctctagcaggctcagttcttccagcacttcaaagcattcagaacgcaatttctgcagcaaatttgcaacgccaaatcaaagtctccacagcaatagacaccactctactgggcaactcttacccaccaaaagatggcgttttcagcaacagtgcaagttcatacataactccaatcataaactttttagccaaaaacggtgccccacttcttgcaaacgtgtacccttacttcgcctacgttaacaatcaacaaaacattggtcttgattatgccttgtttaccaaacaaggcaacaacgaagttgggtaccaaaacctgtttgatgcattggtggattctctgtacgcagctcttgagaaagtgggagcatcaaatgtgaaggttgttgtgtctgagagtgggtggccatcacaaggtggagttggagccactgttcaaaacgcaggaacgtattacaggaatttgatcaaacatgttaagggtggcaccccaaagaggcctaatggacccatagagacttacctctttgccatgtttgatgaaaaccagaagggtggtgcagaaactgagaaacactttggtctcttcaggcctgataaatcaccaaaataccaactcagtttcaattga

SEQ ID NO: 34 - protein, 331 aa, Phaseolus vulgaris
MSALLLLLGVLSSTGVLLTGVESVGVCYGGNGNNLPTKQAVVNLYKSNGIGKIRLYYPDEGALQALRGSNIEVILAVPNDQLQSVSNNGSATNWVNNYVKPYAGNVKLKYIAVGNEVHPGDALAGSVLPALQSIQNAISAANLQRQIKVSTAIDTTLLGNSYPPKDGVFSNSASSYITPIINFLAKNGAPLLANVYPYFAYVNNQQNIGLDYALFTKQGNNEVGYQNLFDALVDSLYAALEKVGASNVKVVVSESGWPSQGGVGATVQNAGTYYRNLIKHVKGGTPKRPNGPIETYLFAMFDENQKGGAETEKHFGLFRPDKSPKYQLSFN

SEQ ID NO: 35 - DNA, 29 bp, Artificial Sequence
  Description of Artificial Sequence Primer
ggaattccgaatctgtgggtgtgtgttat

SEQ ID NO: 36 - DNA, 25 bp, Artificial Sequence
  Description of Artificial Sequence Primer
ggaaacagctatgaccatgattagc

SEQ ID NO: 37 - DNA, 20 bp, Artificial Sequence
  Description of Artificial Sequence Primer
gaggtcacgagttcgtcgta

SEQ ID NO: 38 - DNA, 20 bp, Artificial Sequence
  Description of Artificial Sequence Primer
agctaccatctaaccacggc

SEQ ID NO: 39 - DNA, 20 bp, Artificial Sequence
  Description of Artificial Sequence Primer
tcagctgaggtcacgagttc

SEQ ID NO: 40 - DNA, 22 bp, Artificial Sequence
  Description of Artificial Sequence Primer
tgatcaaacttccccatttagg

SEQ ID NO: 41 - DNA, 22 bp, Artificial Sequence
  Description of Artificial Sequence Primer
tactatgaacccaccacaacag

SEQ ID NO: 42 - DNA, 24 bp, Artificial Sequence
  Description of Artificial Sequence Primer
gaattggacaatgcctgcctgctt

SEQ ID NO: 43 - DNA, 207 bp, Glycine max
atcatcaataactacaacagagtcatatttttatgggaagttgattgcaagggctgcaaggttggtattgattgctgaggagttgaactaccctgatgtgattccaaaggttaggaattttttgaaagaaaccattgagccatggttggagggaacttttagtgggaatggattcctacatgatgaaaaatggggtggcattattac

SEQ ID NO: 44 - DNA, 207 bp, Arabidopsis sp.
ctcagctgaggtcacgagttcgtcgtatttctacgggaaattaatagccagagcagctaggtttgccttaatcgccgaggaagtttgctatctcgatgtgattccgaagattgtaacttacctgaagaacatgattgtgccgtggttagatggtagcttcaaacctaacggctttctgtatgatcctaaatggggaagtttgatcac

SEQ ID NO: 45 - DNA, 186 bp, Glycine max
tcatcacttctgctatgaatccaccacaagagattggtcaaagagtttccaccatcaaaacctttcaggtttcttatcttctgcagtgcactttcattgtcataaaccccttcaagggcatacacaaaccccttccatccttcaccaacaccaccctccctatccaaagcaggcaaagtccactcc

SEQ ID NO: 46 - DNA, 183 bp, Arabidopsis sp.
tcattgtttctactatgaacccaccacaacagattacttaaagagttcccatcatcaaacccatttaatcctttaatcttctccattgctccatctttgtcgtacatactttccaaagcattcacaaatcctttccagccttctccgacgctgtctctagccaaagcaggcattgtccaattc

SEQ ID NO: 47 - DNA, 199 bp, Arabidopsis sp.
gaggtcacgagttcgtcgtatttctacgggaaattaatagccagagcagctaggtttgccttaatcgccgaggaagtttgctatctcgatgtgattccgaagattgtaacttacctgaagaacatgattgagccgtggttagatggtagcttcaaacctaacggctttctgtatgatcctaaatggggaagtttgatca

SEQ ID NO: 48 - DNA, 199 bp, Arabidopsis sp.
gaggtcacgagttcgtcgtatttctacgcgaaattgatcgcgagggcggcgaggttagctttaatagctgaagaagtttgttatctggatgttattccaaagattagaacttacttgaagaacatgatcgagccgtggcttaatggaagtttcggaccaaatggtttcttgtatgatcctaaatggggaagtttgatca

SEQ ID NO: 49 - protein, 66 aa, Arabidopsis sp.
EVTSSSYFYGKLIARAARFALIAEEVCYLDVIPKIVTYLKNMIEPWLDGSFKPNGFLYDPKWGSLI

SEQ ID NO: 50 - protein, 66 aa, Arabidopsis sp.
EVTSSSYFYAKLIARAARLALIAEEVCYLDVIPKIRTYLKNMIEPWLNGSFGPNGFLYDPKWGSLI

SEQ ID NO: 51 - protein, 66 aa, Glycine max
ITTTESYFYGKLIARAARLVLIAEELNYPDVIPKVRNFLKETIEPWLEGTFSGNGFLHDEKWGGII

SEQ ID NO: 52 - protein, 66 aa, Arabidopsis sp.; identical to SEQ ID NO: 50